(19) United States
(12) Patent Application Publication
(10) Pub. No.: US 2009/0170886 A1
(43) Pub. Date: Jul. 2, 2009

(54) ANDROGEN MODULATORS

(75) Inventors: Mark Laurence Boys, Ann Arbor, MI (US); Michelle Marie Bruendl, Ann Arbor, MI (US); Victoria Leigh Downs, Pinckney, MI (US); Stephen Alan Fakhoury, Ann Arbor, MI (US); William Glen Harter, Ann Arbor, MI (US); Lain-Yen Hu, Ann Arbor, MI (US); Sandra Marie Jennings, Ann Arbor, MI (US); Bruce Allen Lefker, Groton, CT (US); Lorna Helen Mitchell, Kent (GB); Raj Kumar Raheja, Ann Arbor, MI (US); Yvonne Dorothy Smith, Ann Arbor, MI (US)

Correspondence Address: PFIZER INC., PATENT DEPARTMENT, Bld 114 M/S 114, EASTERN POINT ROAD, GROTON, CT, 06340, US

(73) Assignee: Pfizer Inc

(21) Appl. No.: 11/997,983

(22) PCT Filed: Jul. 7, 2006
(86) PCT No.: PCT/IB06/02227

§ 371 Date: Oct. 22, 2008

Related U.S. Application Data
(60) Provisional application No. 60/706,413, filed on Aug. 8, 2005.

(51) Int. Cl. A61K 31/435; C07C 43/20; C07D 213/02; A61P 35/00; A61K 31/08

(52) U.S. Cl. 514/277; 568/626; 568/663; 546/339; 514/717

(57) ABSTRACT

The present invention is directed to a new class of benzonitriles and to their use as androgen receptor modulators. Other aspects of the invention are directed to the use of these compounds to decrease excess sebum secretions and to stimulate hair growth.

### FIELD OF THE INVENTION

The present invention is directed to a new class of benzonitrile derivatives and to their use as androgen receptor modulators. Other aspects of the invention are directed to the use of these compounds to decrease sebum secretion and to stimulate hair growth.

### BACKGROUND OF THE INVENTION

Alopecia, or balding, is a common problem which medical science has yet to alleviate. While androgens are associated with balding, the physiological mechanism by which this hair loss occurs is not known. However, it is known that hair growth is altered in individuals afflicted with alopecia.

Hair does not grow continuously but undergoes cycles of activity involving periods of growth, rest, and shedding. The human scalp typically contains from 100,000 to 350,000 hair fibers or shafts, which undergo metamorphosis in three distinct stages:

(a) during the growth phase (anagen) the follicle (i.e. the hair root) penetrates deep into the dermis with the cells of the follicle dividing rapidly and differentiating in the process of synthesizing keratin, the predominant component of hair. In non-balding humans, this growth phase lasts from one to five years;
(b) the transitional phase (catagen) is marked by the cessation of mitosis and lasts from two to three weeks; and
(c) the resting phase (telogen) in which the hair is retained within the scalp for up to 12 weeks, until it is displaced by new follicular growth from the scalp below.

In humans, this growth cycle is not synchronized. An individual will have thousands of follicles in each of these three phases. However, most of the hair follicles will be in the anagen phase. In healthy young adults, the anagen to telogen ratio can be as high as 9 to 1. In individuals with alopecia, this ratio is reduced to as low as 2:1.

Androgenetic alopecia arises from activation of an inherited sensitivity to circulating androgenic hormones. It is the most common type of alopecia. It affects both men (50%) and women (30%), primarily of Caucasian origin. Gradual changes in the width and length of the hair shaft are experienced over time and with increasing age, prematurely in some. Terminal hair is gradually converted to short, wispy, colorless vellus hair. As a consequence, men in their 20's and women in their 30's and 40's begin to notice their hair becoming finer and shorter. In males, most of the hair loss occurs at the crown of the head. Females experience a thinning over their entire scalp. As discussed above, the anagen to telogen ratio is reduced significantly, resulting in less hair growth.

Minoxidil, a potassium channel opener, promotes hair growth. Minoxidil is available commercially in the United States under the trademark, Rogaine While the exact mechanism of action of minoxidil is unknown, its impact on the hair growth cycle is well documented. Minoxidil promotes the growth of the hair follicle and increase the period of time that the hair follicle is in the anagen phase (i.e., increases the anagen to telogen ratio).

While minoxidil promotes hair growth, the cosmetic efficacy of this growth can vary widely. For example, Roenigk reported the results of a clinical trial involving 83 males who used a topical solution of 3% minoxidil for a period of 19 months. Hair growth occurred in 55% of the subjects. However, only 20% of the subjects considered the growth to be cosmetically relevant. (Clin. Res., 33, No. 4, 914A, 1985). Tosti reported cosmetically acceptable re-growth in 18.1% of his subjects. (Dermatologica, 173, No. 3, 136-138, 1986). Thus, the need exists in the art for compounds having the ability to produce higher rates of cosmetically acceptable hair growth in patients with alopecia.

### SUMMARY OF THE INVENTION

In accordance with the present invention, a new class of androgen modulators has been discovered. These compounds, their salts, solvates, and prodrugs thereof, may be represented by Formula I below:

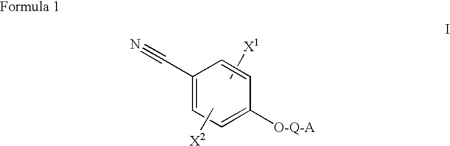

in which;
a) X¹ is represented by halogen, cyano, NO₂, C₁-C₆ alkyl, C₁-C₆ alkoxy or haloalkyl;
b) X² is represented by hydrogen, halogen, cyano, NO₂, C₁-C₆ alkyl, C₁-C₆ alkoxy or haloalkyl;
c) A is represented by:

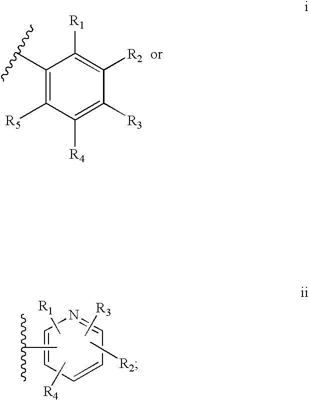

d) Q is represented by C₁-C₆ alkylene which is unsubstituted or optionally substituted with one or more groups each independently selected from:
   i) C₁-C₆ alkyl, optionally substituted;
   ii) C₂-C₆ alkenyl, optionally substituted;
   iii) C₂-C₆ alkynyl, optionally substituted;
   iv) C₃-C₆ cycloalkyl, optionally substituted;
   v) —(C₁-C₆) alkyl(C₆-C₁₀) aryl, in which the alkyl and aryl moieties may each be optionally substituted;
   vi) —(C₆-C₁₀) aryl(C₁-C₆) alkyl, in which the alkyl and aryl moieties may each be optionally substituted; and
   vii) C₁-C₆ alkoxy, optionally substituted;
e) R₁, R₂, R₃, R₄ and R₅ are each independently represented by a substituent selected from the group consisting of:
   i) hydrogen;
   ii) halogen;
   iii) hydroxyl;
   iv) amino;
   v) nitro;
   vi) cyano;
   vii) (C₁-C₁₂)alkyl, optionally substituted;
   viii) (C₁-C₆)alkoxy, optionally substituted;
   ix) (C₃-C₆)cycloalkoxy, optionally substituted;
   x) (C₁-C₃)haloalkyl, optionally substituted;
   xi) (C₂-C₁₂)alkenyl, optionally substituted;
   xii) (C₂-C₁₂)alkynyl, optionally substituted;
   xiii) (C₃-C₁₀)cycloalkyl, optionally substituted;
   xiv) (C₆-C₁₀)aryl, optionally substituted,
   xv) (C₆-C₁₀)aryl(C₁-C₆)alkyl, in which the alkyl and aryl moieties may each be optionally substituted,
   xvi) heteroaryl, optionally substituted;
   xvii) heteroaryl(C₁-C₁₂)alkyl, in which the heteroaryl and alkyl moieties may each be optionally substituted;
   xviii) —O-heterocyclic, optionally substituted;
   xix) heterocyclic(C₁-C₁₂)alkyl-O—, in which the alkyl and heterocyclic moieties may each be optionally substituted;
   xx) —CO₂R₆;
   xxi) —O—COR₆;
   xxii) —CONHR₆;
   xxiii) —NCOR₆; and
   xxiv) —O—(C₁-C₆)alkyl-O—(C₁-C₆)alkyl-O—(C₁-C₆)alkyl; and
f) R₆ is independently hydrogen or C₁-C₆ alkyl;
however, when A is represented by formula i, X¹ or X² is halogen, and Q is methylene, ethylene or n-propylene, A is not

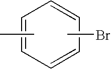

and R₃ is not cyano, bromine, alkynyl, or halogen.

In one embodiment, Q is selected from methylene, ethylene and propylene. Alternately, Q may be methylene. In one embodiment, X² is hydrogen. In another embodiment, one of X¹ or X² is haloalkyl. In yet another embodiment, said haloalkyl is trifluoromethyl. In another embodiment R₁ is represented by hydroxy. In yet another embodiment each of R₁, R₂, R₃, R₄ and R₅ is H. Alternately, A may be phenyl, Q is selected from methylene, ethylene and propylene and one of R₁, R₂, R₃, R₄ and R₅ is hydroxy. Alternately, A may be pyridinyl, Q is selected from methylene, ethylene and propylene and one of R₁, R₂, R₃, and R₄ is hydroxy.

Representative compounds of the present invention include:
4-(1-phenyl-ethoxy)-2-trifluoromethyl-benzonitrile;
(S)-4-(1-phenyl-ethoxy)-2-trifluoromethyl-benzonitrile;
(R)-4-(1-phenyl-ethoxy)-2-trifluoromethyl-benzonitrile;
4-[1-(2-methoxy-phenyl)-ethoxy]-2-t-trifluoromethyl-benzonitrile;
4-[(3-hydroxybenzyl)oxy]-2-(trifluoromethyl)benzonitrile;
4-[1-(3-hydroxyphenyl)ethoxy]-2-(trifluoromethyl)benzonitrile;
(−)-4-[1-(3-hydroxyphenyl)ethoxy]-2-(trifluoromethyl)benzonitrile;
(+)-4-[1-(3-hydroxyphenyl)ethoxy]-2-(trifluoromethyl)benzonitrile;
4-(1-Pyridin-3-yl-ethoxy)-2-trifluoromethyl-benzonitrile;
4-(1-Pyridin-2-yl-ethoxy)-2-trifluoromethyl-benzonitrile;
4-(1-Pyridin-3-yl-ethoxy)-2-trifluoromethyl-benzonitrile;
4-[1-(5-hydroxypyridin-3-yl)ethoxy]-2-(trifluoromethyl)benzonitrile;
(+)4-[1-(5-hydroxypyridin-3-yl)ethoxy]-2-(trifluoromethyl)benzonitrile.

The present invention also comprises the use of a compound of the present invention as a medicine. In another embodiment, the invention relates to the use of a compound in the manufacture of a medicament for inhibiting activation of the androgen receptor. In another embodiment, the invention includes the use of a compound according the invention in the manufacture of a medicament for alleviating a condition selected from the group consisting of hormone dependent cancers, benign hyperplasia of the prostate, acne, hirsutism, excess sebum, alopecia, premenstrual syndrome, lung cancer, precocious puberty, osteoporosis, hypogonadism, age-related decrease in muscle mass, and anemia.

Additionally, the invention includes a pharmaceutical composition comprising a compound of the invention in admixture with one or more pharmaceutically acceptable excipients. The compound of Formula 1 may be prepared as a topical pharmaceutical formulation in admixture with or more pharmaceutically acceptable excipients suitable for dermal application. The compound of formula 1 may be prepared as an article of manufacture, packaged for retail distribution, which advises a consumer how to utilize the compound to alleviate a condition selected from the group consisting of acne, alopecia, and oily skin.

The compounds of Formula I are androgen receptor modulators. The compounds have affinity for the androgen receptor and will cause a biological effect by binding to the receptor. Typically, the compounds will act as antagonists. In selected embodiments they will act as partial agonists, full agonists, or tissue selective agonists. As androgen receptor modulators, the compounds can be used to treat, or alleviate, conditions associated with inappropriate activation of the androgen receptor. Examples of such conditions for antagonists include, but are not limited to, acne, excess sebum secretion, androgenic alopecia, hormone dependant cancers such as prostrate cancer, and hirsutism. Those compounds that are partial agonists, or full agonists, can be used to treat osteoporosis, hypogonadism, anemia, or to stimulate increases in muscle mass, especially in wasting diseases.

The invention is also directed to pharmaceutical compositions containing at least one of the compounds, in an amount effective to modulate activation of the androgen receptor. In a further embodiment, the invention is directed to an article of manufacture containing at least one of the compounds packaged for retail distribution, in association with instructions advising the consumer on how to use the compound to alleviate a condition associated with inappropriate activation of the androgen receptor. An additional embodiment is directed to the use of a compound as a diagnostic agent to detect inappropriate activation of the androgen receptor.

In a further embodiment, the compounds are used topically to induce and/or stimulate hair growth and/or to slow down hair loss. The compounds may also be used topically in the treatment of excess sebum and/or of acne.

In a further embodiment the compounds can be used in livestock such as cattle, pigs, chickens, fish, etc. The compounds will increase the growth rate, and enhance the lean meat to fat ratio in the animals, and improve feed efficiency.

### DETAILED DESCRIPTION OF THE INVENTION

The headings within this document are only being utilized to expedite its review by the reader. They should not be construed as limiting the invention or claims in any manner.

### DEFINITIONS AND EXEMPLIFICATION

As used throughout this application, including the claims, the following terms have the meanings defined below, unless specifically indicated otherwise. The plural and singular should be treated as interchangeable, other than the indication of number:
a. “halogen” refers to a chlorine, fluorine, iodine or bromine atom.
b. “C₁-C₆ alkyl” refers to a branched or straight chained alkyl group containing from 1 to 6 carbon atoms, such as methyl, ethyl, n-propyl, isopropyl, n-butyl, isobutyl, pentyl, etc.
c. “C₁-C₆ alkyl, optionally substituted” refers to a branched or straight chained alkyl group containing from 1 to 6 carbon atoms, such as methyl, ethyl, n-propyl, isopropyl, n-butyl, isobutyl, pentyl, etc. Such an alkyl group may be optionally substituted, in which up to 6 hydrogen atoms are replaced by a substituent selected from the group consisting of halogen, haloalkyl, hydroxy, thiol, cyano, and NR₆R₇ in which each R₆ and R₇ are independently represented by hydrogen or C₁-C₆ alkyl.
d. “C₁-C₁₂ alkyl, optionally substituted” refers to a branched or straight chained alkyl group containing from 1 to 12 carbon atoms, such as methyl, ethyl, n-propyl, isopropyl, n-butyl, isobutyl, hexyl, octyl, decyl, etc. Such an alkyl group may be optionally substituted, in which up to 8 hydrogen atoms are replaced by a substituent selected from the group consisting of halogen, haloalkyl, hydroxy, thiol, cyano, and NR₆R₇ in which each R₆ and R₇ are independently represented as defined above.
e. “C₁-C₆ alkylene” refers to a bivalent straight chained alkyl radical containing from 1 to 6 carbon atoms, such as methylene, ethylene, n-propylene, n-butylene, pentylene, etc
f. “C₂-C₆ alkenyl” refers to a straight-chain or branched-chain hydrocarbon radical containing from 2 to 6 carbon atoms and 1, or more, carbon-carbon double bonds. Examples of alkenyl radicals include ethenyl, propenyl, 1,4-butadienyl, 1-hexenyl, 1,3-octadienyl, and the like.
g. C₂-C₆ alkenyl, optionally substituted” refers to a straight-chain or branched-chain hydrocarbon radical containing from 2 to 6 carbon atoms and 1, or more, carbon-carbon double bonds. Such an alkenyl group may be optionally substituted, in which up to 8 hydrogen atoms, where chemically permissible, are replaced by a substituent selected from the group consisting of halogen, haloalkyl, hydroxy, thiol, cyano, and NR₆R₇ in which R₆ and R₇ are as defined above. Examples of “substituted alkenyl radicals” include, but are not limited to, propen-2-ol, prop-2-en-1-ol, 5-phloro-pent-2-en-3-ol, and 5-phloro-hexa-2,5-dien-3-ol.
h. “C₂-C₁₂ alkenyl” refers to a straight-chain or branched-chain hydrocarbon radical containing from 2 to 12 carbon atoms and 1, or more, carbon-carbon double bonds. The term “C₂-C₁₂ alkenyl” encompasses any number of carbon atoms from 2 to 12 having one or more carbon-carbon double bond. Examples of C₂-C₁₂ alkenyl radicals include ethenyl, propenyl, 1,4-butadienyl, 1-hexenyl, 1,3-octadienyl and the like.
i. “C₂-C₁₂ alkenyl, optionally substituted” refers to a straight-chain or branched-chain hydrocarbon radical containing from 2 to 12 carbon atoms and 1, or more, carbon-carbon double bonds. Such an alkenyl group may be optionally substituted, in which up to 8 hydrogen atoms are replaced by a substituent selected from the group consisting of halogen, haloalkyl, hydroxy, thiol, cyano, and NR₆R₇ in which R₆ and R₇ are as defined above. Examples of substituted C₂-C₁₂ alkenyl radicals include ethenyl, propenyl, 1,4-butadienyl, 1-hexenyl, 1,3-octadienyl and the like. Examples of substituted alkenyl radicals include, but are not limited to, 1-propyl-hexa-3,5-dienylamine, 7-amino-hept-5-en-3-ol, 5-fluoromethyl-hept-2-enylamine, etc.
j. “C₂-C₆ alkynyl” refers to a straight-chain or branched-chain hydrocarbon radical containing from 2 to 6 carbon atoms and having 1, or more, carbon-carbon triple bonds. Examples of alkynyl radicals include ethynyl, propynyl, butynyl, octynyl, and the like. Such an alkynyl group may be optionally substituted, in which up to 8 hydrogen atoms, where chemically possible, are replaced by a substituent selected from the group consisting of halogen, hydroxy, haloalkyl, thiol, cyano, and —NR₆R₇ in which R₆ and R₇ are as defined above.
k. “C₂-C₆ alkynyl optionally substituted” refers to a straight-chain or branched-chain hydrocarbon radical containing from 2 to 6 carbon atoms and having 1, or more, carbon-carbon triple bonds. Examples of alkynyl radicals include ethynyl, propynyl, butynyl, octynyl, and the like. Such an alkynyl group may be optionally substituted, in which up to 8 hydrogen atoms, where chemically possible, are replaced by a substituent selected from the group consisting of halogen, hydroxy, haloalkyl, thiol, cyano, and —NR₆R₇ in which R₆ and R₇ are as defined above. Examples of substituted C₂-C₆ alkynyl radicals include, but are not limited to, 4-chloro-hex-2-yne, and 5-fluoromethyl-hept-2-enylamine.
l. “C₂-C₁₂ alkynyl optionally substituted” refers to a straight-chain or branched-chain hydrocarbon radical containing from 2 to 12 carbon atoms and having 1, or more, carbon-carbon triple bonds. Examples of alkynyl radicals include ethynyl, propynyl, butynyl, octynyl, and the like. Such an alkynyl group may be optionally substituted, in which up to 8 hydrogen atoms are replaced by a substituent selected from the group consisting of halogen, hydroxy, haloalkyl, thiol, cyano, and —NR₆R₇ in which R₆ and R₇ are as defined above. Examples of substituted C₂-C₁₂ alkynyl radicals include, but are not limited to, 4-chloro-hex-2-yne, 5-fluoromethyl-hept-2-enylamine, 5-fluoromethyl-hept-2-ynylamine, (5,5,5-frifluoro-4-methyl-pent-2-ynyl)-hydrazine and the like.
m. “haloalkyl” refers to a branched or straight chained alkyl group containing from 1 to 6 carbon atoms, in which at least one hydrogen atom is replaced with a halogen (i.e., C₁-C₃ haloalkyl, C₁-C₆ haloalkyl). Examples of suitable haloalkyls include chloromethyl, difluoromethyl, trifluoromethyl, 1-fluoro-2-chloro-ethyl, 5-fluoro-hexyl, 3-difluoro-isopropyl, 3-chloro-isobutyl, etc.
n. “(C₁-C₂)alkyl substituted with one or more halogen atoms” refers to a straight chained alkyl group containing 1 or 2 carbon atoms, i.e., methyl or ethyl in which at least one hydrogen atom is replaced with a halogen (i.e. for example trifluoromethyl, dichloromethyl, etc.).
o. “(C₁-C₂)alkoxy substituted with one or more halogen atoms” refers to a straight chained alkoxy group containing 1 or 2 carbon atoms, i.e., methoxy or ethoxy in which at least one hydrogen atom is replaced with a halogen (i.e. for example trifluoromethoxy, difluoromethoxy, etc.)
p. “C₁-C₆ alkoxy” refers to a straight or branched chain alkoxy group containing from 1 to 6 carbon atoms, such as methoxy, ethoxy, n-propoxy, isopropoxy, n-butoxy, isobutoxy, pentoxy, etc.
q. “C₁-C₆ alkoxy” optionally substituted, refers to a straight or branched chain alkoxy group containing from 1 to 6 carbon atoms, such as methoxy, ethoxy, n-propoxy, isopropoxy, n-butoxy, isobutoxy, pentoxy, etc. wherein at least one hydrogen atom is replaced by a substituent selected from the group consisting of halogen, haloalkoxy, C₁-C₆ alkyl, etc.
r. “haloalkoxy” refers to a branched or straight chained alkoxy group containing from 1 to 6 carbon atoms, in which at least one hydrogen atom is replaced with a halogen (i.e. C₁-C₆ haloalkoxy). Examples of suitable haloalkoxys include chloromethoxy, difluoromethoxy, trifluoromethoxy, 1-fluoro-2-chloro-ethoxy, 5-fluoro-hexoxy, 3-difluoro-isopropoxy, 3-chloro-isobutoxy, etc.
s. “(C₆-C₁₀)aryl” optionally substituted means a cyclic, aromatic hydrocarbon containing from 6 to 10 carbon atoms. Examples of aryl groups include phenyl, naphthyl and biphenyl. Such an aryl moiety may be optionally substituted with up to 4 non-hydrogen substituents, each substituent is independently selected from the group consisting of halogen, nitro, cyano, hydroxy, (C₁-C₆)alkyl, (C₁-C₆)alkoxy, (C₁-C₂)alkyl substituted with one or more halogens, (C₁-C₂)alkoxy substituted with one or more halogens, —C(O)—R₆, —C(O)—O—R₆, SR₆ SO₂R₆ and NR₆. R₆ is represented by C₁-C₆ alkyl or hydrogen. These substituents may be the same or different and may be located at any position of the ring, that is chemically permissible.
t. “(C₃-C₆) cycloalkyl” refers to a saturated or partially saturated monocyclic, bicyclic or tricyclic alkyl radical wherein each cyclic moiety has 3 to 6 carbon atoms. Examples of cycloalkyl radicals include cyclopropyl, cyclobutyl, cyclopentyl, and cyclohexyl. Such a cycloalkyl group may be optionally substituted, in which up to 4 hydrogen atoms are replaced by a substituent selected from the group consisting of halogen, cyano, nitro, hydroxy, (C₁-C₆)alkyl, (C₁-C₆)alkoxy, (C₁-C₂)alkyl substituted with one or more halogens, (C₁-C₂)alkoxy substituted with one or more halogens, —C(O)—R₆, —C(O)—O—R₆, SR₆, SO₂R₆ and NR₆R₇ in which R₆ and R₇ are as defined above.
u. “(C₃-C₆) cycloalkyl” optionally substituted, refers to a saturated or partially saturated monocyclic, bicyclic or tricyclic alkyl radical wherein each cyclic moiety has 3 to 6 carbon atoms, in which up to 4 hydrogen atoms are replaced by a substituent selected from the group consisting of halogen, cyano, nitro, hydroxy, (C₁-C₆)alkyl, (C₁-C₆)alkoxy, (C₁-C₂)alkyl substituted with one or more halogens, (C₁-C₂)alkoxy substituted with one or more halogens, —C(O)—R₆, —C(O)—O—R₆, SR₆, SO₂R₆ and NR₆R₇ in which R₆ and R₇ are as defined above.
v. “(C₃-C₁₀) cycloalkyl” optionally substituted refers to a saturated or partially saturated monocyclic, bicyclic or tricyclic alkyl radical wherein each cyclic moiety has 3 to 10 carbon atoms. Examples of cycloalkyl radicals include cyclopropyl, cyclobutyl, cyclopentyl, cyclohexyl, cyclooctyl, and the like. Such a cycloalkyl group may be optionally substituted, in which up to 4 hydrogen atoms are replaced by a substituent selected from the group consisting of halogen, cyano, nitro, hydroxy, (C₁-C₆)alkyl, (C₁-C₆)alkoxy, (C₁-C₂)alkyl substituted with one or more halogens, (C₁-C₂)alkoxy substituted with one or more halogens, —C(O)—R₆, —C(O)—O—R₆, SR₆, SO₂R₆ and NR₆R₇ in which R₆ and R₇ are as defined above.
w. “heteroaryl” refers to an aromatic ring having one, or more, heteroatoms selected from oxygen, nitrogen and sulfur. More specifically, it refers to a 5- or 6-membered ring containing 1, 2, 3, or 4 nitrogen atoms; 1 oxygen atom; 1 sulfur atom; 1 nitrogen and 1 sulfur atom; 1 nitrogen and 1 oxygen atom; 2 nitrogen atoms and 1 oxygen atom; or 2 nitrogen atoms and 1 sulfur atom. The 5-membered ring has 2 double bonds and the 6-membered ring has 3 double bonds. The term heteroaryl also includes bicyclic groups in which the heteroaryl ring is fused to a benzene ring, heterocyclic ring, a cycloalkyl ring, or another heteroaryl ring. Examples of such heteroaryl ring systems include, but are not limited to, pyrrolyl, furanyl, thienyl, imidazolyl, oxazolyl, indolyl, thiazolyl, pyrazolyl, pyridinyl, pyrimidinyl, purinyl, quinolinyl, benzofuran, tetrazole, isoquinolinyl, oxadiazolyl, thiadiazolyl, isothiazolyl, isoxazolyl, triazolyl, benzo[b]thienyl, 2-, 4-, 5-, 6-, or 7-benzoxazolyl, 7-benzimidazolyl, or benzothiazolyl.
x. “heteroaryl, optionally substituted,” refers to a heteroaryl moiety as defined immediately above, in which up to 4 carbon atoms of the heteroaryl moiety may be substituted with a substituent, each substituent is independently selected from the group consisting of halogen, cyano, nitro, hydroxy, (C₁-C₆)alkyl, (C₁-C₆)alkoxy, (C₁-C₂)alkyl substituted with one or more halogens, (C₁-C₂)alkoxy substituted with one or more halogens, SO₂R₆—C(O)—R₆, —C(O)—O—R₆, SR₆, and NR₆, in which R₆ is as defined above.
y. “heterocycle” or “heterocyclic ring” refers to any 3- or 4-membered ring containing a heteroatom selected from oxygen, nitrogen and sulfur; or a 5-, 6-, 7-, 8-, 9-, or 10-membered ring containing 1, 2, or 3 nitrogen atoms; 1 oxygen atom; 1 sulfur atom; 1 nitrogen and 1 sulfur atom, 1 nitrogen and 1 oxygen atom; 2 oxygen atoms in non-adjacent positions; 1 oxygen and 1 sulfur atom in non-adjacent positions; or 2 sulfur atoms in non-adjacent positions. The 5-membered ring has 0 to 1 double bonds, the 6- and 7-membered rings have 0 to 2 double bonds, and the 8, 9, or 10-membered rings may have 0, 1, 2, or 3 double bonds. The term “heterocyclic” also includes bicyclic groups in which any of the above heterocyclic rings is fused to a benzene ring, a cyclohexane or cyclopentane ring or another heterocyclic ring (for example, indolyl, quinolyl, isoquinolyl, tetrahydroquinolyl, benzofuryl, dihydrobenzofuryl or benzothienyl and the like). Heterocyclics include: pyrrolidinyl, tetrahydrofuranyl, tetrahydrothiophenyl, piperidinyl, piperazinyl, azepane, azocane, morpholinyl, isochromyl, quinolinyl, tetrahydrotriazine, tetrahydropyrazole, dihydro-oxathiol-4-yl, dihydro-1H-isoindole, tetrahydro-oxazolyl, tetrahydro-oxazinyl, thiomorpholinyl, tetrahydropyrimidinyl, dioxolinyl, octahydrobenzofuranyl, octahydrobenzimidazolyl, and octahydrobenzothiazolyl.
z. “heterocyclic, optionally substituted” refers to a heterocyclic moiety as defined immediately above, in which up to 4 carbon atoms of the heterocycle moiety may be substituted with a substituent, each substituent is independently selected from the group consisting of halogen, cyano, nitro, hydroxy, (C₁-C₆)alkyl, (C₁-C₆)alkoxy, (C₁-C₂)alkyl substituted with one or more halogens, (C₁-C₂)alkoxy substituted with one or more halogens, —C(O)—R₆, —C(O)—O—R₆, SR₆, SO₂R₆ and NR₆R₇ in which R₆ and R₇ are as defined above. Any nitrogen atom within such a heterocyclic ring may optionally be substituted with (C₁-C₆) alkyl, if such substitution is chemically permissible.
aa. “androgen” refers to testosterone and its precursors and metabolites, and 5-alpha reduced androgens, including but not limited to dihydrotestosterone. Androgen refers to androgens from the testis, adrenal gland, and ovaries, as well as all forms of natural, synthetic and substituted or modified androgens.
bb. “pharmaceutically acceptable” means suitable for use in mammals.
cc. “salts” is intended to refer pharmaceutically acceptable salts and to salts suitable for use in industrial processes, such as the preparation of the compound.
dd. “pharmaceutically acceptable salts” is intended to refer to either “pharmaceutically acceptable acid addition salts” or “pharmaceutically acceptable basic addition salts” depending upon actual structure of the compound.
ee. “pharmaceutically acceptable acid addition salts” is intended to apply to any non-toxic organic or inorganic acid addition salt of the base compounds represented by Formula I or any of its intermediates. Illustrative inorganic acids which form suitable salts include hydrochloric, hydrobromic, sulphuric, and phosphoric acid and acid metal salts such as sodium monohydrogen orthophosphate, and potassium hydrogen sulfate. Illustrative organic acids, which form suitable salts include the mono-, di-, and tricarboxylic acids. Illustrative of such acids are for example, acetic, glycolic, lactic, pyruvic, malonic, succinic, glutaric, fumaric, malic, tartaric, citric, ascorbic, maleic, hydroxymaleic, benzoic, hydroxy-benzoic, phenylacetic, cinnamic, salicylic, 2-phenoxybenzoic, p-toluenesulfonic acid, and sulfonic acids such as methane sulfonic acid and 2-hydroxyethane sulfonic acid. Such salts can exist in either a hydrated or substantially anhydrous form. In general, the acid addition salts of these compounds are soluble in water and various, hydrophilic organic solvents, and which in comparison to their free base forms, generally demonstrate higher melting points.
ff. “pharmaceutically acceptable basic addition salts” is intended to apply to any non-toxic organic or inorganic basic addition salts of the compounds represented by Formula I, or any of its intermediates. Illustrative bases which form suitable salts include alkali metal or alkaline-earth metal hydroxides such as sodium, potassium, calcium, magnesium, or barium hydroxides; ammonia, and aliphatic, alicyclic, or aromatic organic amines such as methylamine, dimethylamine, trimethylamine, and picoline.
gg. “prodrug” refers to compounds that are rapidly transformed in vivo to yield the parent compound of the above formulas, for example, by hydrolysis in blood. A thorough discussion is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are incorporated herein by reference.
hh. “compound of Formula I”, “compounds of the invention”, and “compounds” are used interchangeably throughout the application and should be treated as synonyms.
ii. “patient” refers to warm blooded animals such as, for example, guinea pigs, mice, rats, gerbils, cats, rabbits, dogs, monkeys, chimpanzees, stump tail macaques, and humans.
jj. “treat” refers to the ability of the compounds to either relieve, alleviate, or slow the progression of the patient's disease (or condition) or any tissue damage associated with the disease.
kk. “livestock” refers to animals suitable for human meat consumption. Examples include pigs, cattle, chickens, fish, turkeys, rabbits, etc.
ll. “isomer” means “stereoisomer” and “geometric isomer” as defined below.
mm. “stereoisomer” means compounds that possess one or more chiral centers and each center may exist in the R or S configuration. Stereoisomers include all diastereomeric, enantiomeric and epimeric forms as well as racemates and mixtures thereof.
nn. “geometric isomer” means compounds that may exist in cis, trans, anti, entgegen (E), and zusammen (Z) forms as well as mixtures thereof.

Certain of the compounds of the formula (I) may exist as geometric isomers. The compounds of the formula (I) may possess one or more asymmetric centers, thus existing as two, or more, stereoisomeric forms. The present invention includes all the individual stereoisomers and geometric isomers of the compounds of formula (I) and mixtures thereof. Individual enantiomers can be obtained by chiral separation or using the relevant enantiomer in the synthesis.

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention. The compounds may also exist in one or more crystalline states, i.e. polymorphs, or they may exist as amorphous solids. All such forms are encompassed by the claims.

All of the compounds of Formula I contain a benzonitrile moiety. To further exemplify the invention, the numbering system for this ring and its substitution pattern is shown below:

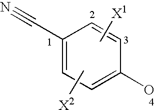

Position 1 of this benzonitrile is substituted with a cyano moiety as depicted above. Position 4 is substituted with an oxygen atom forming an ether moiety. The benzonitrile will be further substituted, as depicted by X¹, at any of position 2, 3, 5 or 6 with a halogen atom, a cyano group, a (C₁-C₆) alkyl group, a nitro, or a haloalkyl moiety. Typically, it will be a halogen or haloalkyl moiety located at the 2- or 6-position. More typically, it will be trifluoromethyl located at the 2, 3, 5 or 6-position of the benzonitrile. The benzonitrile may optionally be further substituted, as indicated by X², with a third substituent, selected from the group consisting of halogen, cyano, (C₁-C₆) alkyl, a nitro, and haloalkyl which may be located at any position of the benzonitrile not substituted by another moiety.

All of the compounds of Formula I contain at least one phenyl moiety (ring i) or a pyridyl moiety (ring ii), which moieties of rings i or ii may be unsubstituted or optionally substituted as described above. To further exemplify the invention, the numbering system for ring i is shown below.

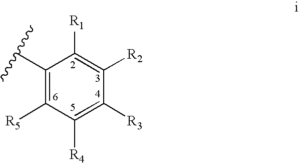

The phenyl moiety may be bonded to the methylene, ethylene or n-propylene moiety at any of positions 2, 3, 4, 5, or 6. The phenyl may be further optionally substituted at one or more of the remaining positions as indicated by the R₁, R₂, R₃, R₄ and R₅ moieties. Any of positions 2, 3, 4, 5, or 6 may be substituted (if chemically permissible).

In a second embodiment, the pyridyl moiety is as shown below (i.e., ring ii):

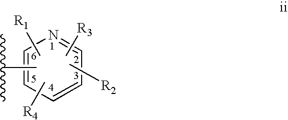

A nitrogen atom is located at position 1 of the pyridine moiety. The pyridine ring may be optionally independently substituted at positions 2 through 6 with one or more of the entities listed above for R₁, R₂, R₃, R₄ and R₅ moieties. The pyridyl may be bonded to the methylene, ethylene or n-propylene moiety at any of positions 2, 3, 4, 5 or 6. The pyridyl may be further substituted at the remaining positions as indicated by any of the R₁, R₂, R₃, and R₄ moieties. Any of positions 2, 3, 4, 5, or 6 may be mono-substituted, or di-substituted (if chemically permissible).

More specific embodiments of the invention include compounds of Formula I in which:
i) X¹ is chloro or trifluoromethyl and is located at the 2-position of the phenyl ring, and X² is hydrogen;
ii) X¹ is chloro or trifluoromethyl and is located at the 2-position of the phenyl ring, and X² is hydrogen and Q is methylene;
iii) X¹ is trifluoromethyl and is located at the 2-position of the phenyl ring, X² is hydrogen, and Q is methylene;
iv) X¹ is trifluoromethyl and is located at the 2-position of the phenyl ring, X² is hydrogen, Q is methylene, and A is represented by ring i;
v) X¹ is trifluoromethyl and is located at the 2-position of the phenyl ring, X² is hydrogen, Q is methylene, and A is represented by ring ii.

### Synthesis

The compounds of Formula I can be prepared using methods known in the art for the preparation of ethers. The reader's attention is directed to European Patent Application Number 58932, published Sep. 1, 1982, for a generalized description of the preparation aryl ethers.

Scheme I below provides an overview of one such technique for preparing compounds in which A is represented by ring i or ring ii.

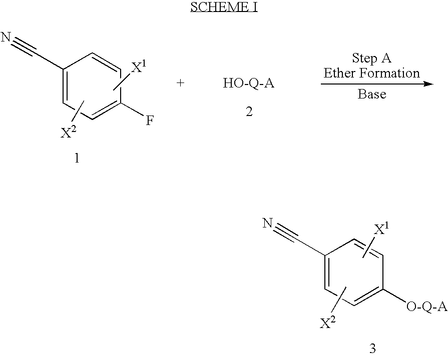

As depicted above, one of the starting materials for Step A is a 4-fluoro-benzonitrile as depicted by structure 1. X¹ and X² should each be represented by the same substituents as desired in the final product. These benzonitriles are known in the art and may be purchased commercially or may be synthesized by methods known in the art. See, for instance, Organic Letters, 6(17), 2837-2840, 2004; Journal of Organometallic Chemistry, 684 (1-2), 50-55, 2003; Journal of European Chemistry, 45 (18)3597-3603, 1980; Japanese Kokai Tokkyo Koho, 2001097937; European Patent Application No. 534317 and European Patent Application No. 1266904.

The other starting material for Step A is an alcohol as depicted by structure 2. Q-A should be represented by the same substituent(s) as is desired in the final product. Such phenyl alkanols or pyridinyl alkanols are known in the art. Many may be purchased from known commercial sources. Alternatively, they can be prepared as described in Archiv der Pharmazie (Weinheim, Germany), 308(5), 325-331, 1975.

In Step A, the benzonitrile ether phenyl alkanol or pyridinyl alkanol of structure 3 is produced via a nucleophilic substitution as is known in the art. The alcohol of structure 2 is contacted with a slight excess of a base, such as sodium hydride, potassium t-butoxide, etc., to produce an alkoxide ion. The reaction is carried out in an aprotic solvent, such as tetrahydrofuran, typically, under an inert atmosphere (typically nitrogen) at a temperature of about 0° C. The alcohol is stirred with the base for a period of time ranging from 5 minutes to 8 hours, typically from about 5 minutes to 60 minutes.

One equivalent of the 4-fluoro-benzonitrile of structure 1 is then added to the reaction medium and the reactants are stirred for a sufficient period of time to allow the alkoxide ion to displace the fluorine from the benzonitrile. This typically takes from 30 minutes to 24 hours. The reaction is typically allowed to warm to room temperature.

Alternatively, the alcohol of structure 2 and the fluorobenzonitrile are combined in one reaction vessel and contacted with a slight excess of a base, such as sodium hydride, potassium t-butoxide, etc., to produce an alkoxide ion. The reaction is carried out under the conditions described above to form the compound of structure 3.

The resulting compound depicted by structure 3 can be recovered by extraction, evaporation, or other techniques known in the art. It may optionally be purified by chromatography, recrystallization, distillation, or other techniques known in the art prior.

Alternatively, the etherification can be carried out using a weak base such as sodium hydroxide, potassium hydroxide, lithium hydroxide, magnesium hydroxide, sodium carbonate, potassium carbonate, cesium carbonate, potassium phosphate, sodium phosphate, potassium phosphonate, sodium phosphonate, sodium bicarbonate, etc. Reactions with weak bases are typically carried out under hydrous conditions (i.e. an admixture of water and an organic solvent such as dimethylformamide, tetrahydrofuran, etc.). Alternatively, the reaction may be carried out with a weak base in an aprotic solvent under anhydrous conditions. The 4-fluoro-benzonitrile of structure 1 and the alcohol of structure of 2 are contacted in the presence of the base at a temperature ranging from room temperature to reflux.

Certain steps in the synthesis of these compounds may utilize a reaction to protect certain reactive groups during the synthesis and to ensure that the reaction takes place at the desired reactive group. Such protective steps are well known in the art.

The deprotection reaction will vary depending upon the identity of the protecting group. For example, if a benzyl protecting group is utilized, it may be removed by contacting it with trifluoracetic acid and triethylsilane under heat. Other protecting groups may be used. The reader's attention is directed to T. W. Greene, Protective Groups in Organic Synthesis, John Wiley & Sons, New York, 1991 for a further discussion of potential protecting groups and their removal.

The reaction scheme described above applies equally to those compounds in which A is represented by ring (ii), i.e. a substituted or unsubstituted pyridinyl moiety. The only modification required pertains to one of the starting materials utilized in Step A.

As would be appreciated by those skilled in the art, some of the methods useful for the preparation of such compounds, as discussed above, may require protection of a particular functionality, e.g., to prevent interference by such functionality in reactions at other sites within the molecule or to preserve the integrity of such functionality. The need for, and type of, such protection is readily determined by one skilled in the art, and will vary depending on, for example, the nature of the functionality and the conditions of the selected preparation method. See, e.g., T. W. Greene, supra.

Some of the compounds of this invention are acidic and they form salts with pharmaceutically acceptable cations. Some of the compounds of this invention are basic and form salts with pharmaceutically acceptable anions. All such salts are within the scope of this invention and they can be prepared by conventional methods such as combining the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate. The compounds are obtained in crystalline form according to procedures known in the art, such as by dissolution in an appropriate solvent(s) such as ethanol, hexanes or water/ethanol mixtures.

### Medical and Cosmetic Uses

The compounds of Formula I are androgen receptor modulators. They can be used to alleviate conditions associated with inappropriate activation of the androgen receptor. Compounds acting as androgen antagonists may be used to treat, or alleviate, hormone dependent cancers such as prostate carcinomas, benign hyperplasia of the prostate, acne, hirsutism, excess sebum, alopecia, hypertrichosis, precocious puberty, prostamegaly, virilization, and polycystic ovary syndrome. Compounds acting as partial agonists, or full agonists, may be used to treat, or alleviate, male hypogonadism, male sexual dysfunction (impotence, male dysspemtatogenic sterility), abnormal sex differentiation (male hermaphroditism), male delayed puberty, male infertility, aplastic anemia, hemolytic anemia, sickle cell anemia, idiopathic thrombocytopenic purpura, myelofibrosis, renal anemia, wasting diseases (post operative, malignant tumor, trauma, chronic renal disease, burn or AIDS induced), abatement of pain in terminal carcinoma of female genitalia, inoperable breast cancer, mastopathy, endometriosis, female sexual dysfunction, osteoporosis, wound healing and muscle tissue repair.

In order to exhibit the therapeutic properties described above, the compounds need to be administered in a quantity sufficient to modulate activation of the androgen receptor. This amount can vary depending upon the particular disease/condition being treated, the severity of the patient's disease/condition, the patient, the particular compound being administered, the route of administration, and the presence of other underlying disease states within the patient, etc. When administered systemically, the compounds typically exhibit their effect at a dosage range of from about 0.1 mg/kg/day to about 100 mg/kg/day for any of the diseases or conditions listed above. Repetitive daily administration may be desirable and will vary according to the conditions outlined above.

The compounds of the present invention may be administered by a variety of routes. They may be administered orally. The compounds may also be administered parenterally (i.e., subcutaneously, intravenously, intramuscularly, intraperitoneally, or intrathecally), rectally, or topically.

In a typical embodiment, the compounds are administered topically. Topical administration is especially appropriate for hirsutism, alopecia, acne and excess sebum. The dose will vary, but as a general guideline, the compound will be present in a dermatologically acceptable carrier in an amount of from about 0.01 to 50 w/w %, and more typically from about 0.1 to 10 w/w %. The dermatological preparation will be applied to the affected area from 1 to 4 times daily. “Dermatologically acceptable” refers to a carrier which may be applied to the skin or hair, and which will allow the drug to diffuse to the site of action. More specifically, it refers the site where inhibition of activation of an androgen receptor is desired.

In a further embodiment, the compounds are used topically to relieve alopecia, especially androgenic alopecia. Androgens have a profound effect on both hair growth and hair loss. In most body sites, such as the beard and pubic skin, androgens stimulate hair growth by prolonging the growth phase of the hair cycle (anagen) and increasing follicle size. Hair growth on the scalp does not require androgens but, paradoxically, androgens are necessary for balding on the scalp in genetically predisposed individuals (androgenic alopecia) where there is a progressive decline in the duration of anagen and in hair follicle size. Androgenic alopecia is also common in women where it usually presents as a diffuse hair loss rather than showing the patterning seen in men.

While the compounds will most typically be used to alleviate androgenic alopecia, the invention is not limited to this specific condition. The compounds may be used to alleviate any type of alopecia. Examples of non-androgenic alopecia include alopecia greata, alopecia due to radiotherapy or chemotherapy, scarring alopecia, stress related alopecia, etc. As used in this application, “alopecia” refers to partial or complete hair loss on the scalp.

Thus, the compounds can be applied topically to the scalp and hair to prevent, or alleviate balding. Further, the compound can be applied topically in order to induce or promote the growth of hair on the scalp.

In a further embodiment of the invention, a compound of Formula I is applied topically in order to prevent the growth of hair in areas where such hair growth is not desired. One such use will be to alleviate hirsutism. Hirsutism is excessive hair growth in areas that typically do not have hair (i.e. a female face). Such inappropriate hair growth occurs most commonly in women and is frequently seen at menopause. The topical administration of the compounds will alleviate this condition leading to a reduction, or elimination of this inappropriate, or undesired, hair growth.

The compounds may also be used topically to decrease sebum production. Sebum is composed of triglycerides, wax esters, fatty acids, sterol esters and squalene. Sebum is produced in the acinar cells of the sebaceous glands and accumulates as these cells age. At maturation, the acinar cells lyse, releasing sebum into the lumenal duct so that it may be deposited on the surface of the skin.

In some individuals, an excessive quantity of sebum is secreted onto the skin. This can have a number of adverse consequences. It can exacerbate acne, since sebum is the primary food source for Propionbacterium acnes, the causative agent of acne. It can cause the skin to have a greasy appearance, typically considered cosmetically unappealing.

Formation of sebum is regulated by growth factors and a variety of hormones including androgen. The cellular and molecular mechanism by which androgens exert their influence on the sebaceous gland has not been fully elucidated. However, clinical experience documents the impact androgens have on sebum production. Sebum production is significantly increased during puberty, when androgen levels are their highest. Anti-androgens, such as finasteride, have been shown to decrease androgen secretion. For additional information on sebum production and androgens role in skin metabolism, see Moshell et al, Progress in Dermatology, vol. 37, No. 4, December 2003.

Thus, the compounds of formula I inhibit the secretion of sebum and thus reduce the amount of sebum on the surface of the skin. The compounds can be used to treat a variety of dermal diseases such as acne or seborrheic dermatitis.

In addition to treating diseases associated with excess sebum production, the compounds can also be used to achieve a cosmetic effect. Some consumers believe that they are afflicted with overactive sebaceous glands. They feel that their skin is oily and thus unattractive. These individuals can utilize the compounds of Formula I to decrease the amount of sebum on their skin. Decreasing the secretion of sebum will alleviate oily skin in individuals afflicted with such conditions.

The compounds may also be used to treat sebaceous hyperplasia. Sebaceous hyperplasia is the term used for enlarged sebaceous glands seen on the skin of the middle-aged and elderly. Most typically they occur on the forehead or cheeks. While these enlarged glands are not harmful, many individuals feel that they are cosmetically unattractive. Isotretinoin, which reduces sebum secretion, has been shown to reduce the size of these enlarged glands. Thus, by reducing sebum secretion, these compounds will also alleviate sebaceous hyperplasia.

In a further embodiment, those compounds acting as partial agonists, or full agonists, may be used to treat, or alleviate, osteoporosis. Osteoporosis is characterized by bone loss, resulting from an imbalance between bone resorption (destruction) and bone formation, which starts in the fourth decade and continues throughout life at the rate of about 1-4% per year (Eastell, Treatment of postmenopausal osteoporosis, New Eng. J. Med. 338: 736, 1998). In the United States, there are currently about 20 million people with detectable fractures of the vertebrae due to osteoporosis. In addition, there are about 250,000 hip fractures per year due to osteoporosis, associated with a 12%-20% mortality rate within the first two years, while 30% of patients require nursing home care after the fracture and many never become fully ambulatory again. In postmenopausal women, estrogen deficiency leads to increased bone resorption resulting in bone loss in the vertebrae of around 5% per year, immediately following menopause. Thus, first line treatment/prevention of this condition is inhibition of bone resorption by bisphosphonates, estrogens, selective estrogen receptor modulators (SERMs) and calcitonin. However, inhibitors of bone resorption are not sufficient to restore bone mass for patients who have already lost a significant amount of bone. The increase in spinal BMD attained by bisphosphonate treatment can reach 11% after 7 years of treatment with alendronate. In addition, as the rate of bone turnover differs from site to site; higher in the trabecular bone of the vertebrae than in the cortex of the long bones, the bone resorption inhibitors are less effective in increasing hip BMD and preventing hip fracture. Therefore, osteoanabolic agents, which increase cortical/periosteal bone formation and bone mass of long bones, would address an unmet need in the treatment of osteoporosis especially for patients with high risk of hip fractures.

A number of studies demonstrate that androgens are osteoanabolic in women and men. Anabolic steroids, such as nandrolone decanoate or stanozolol, have been shown to increase bone mass in postmenopausal women. Beneficial effects of androgens on bone in post-menopausal osteoporosis are well documented in recent studies using combined testosterone and estrogen administration (Hofbauer, et al., Androgen effects on bone metabolism: recent progress and controversies, Eur. J. Endocrinol. 140, 271-286, 1999). Thus those compounds of Formula I exhibiting agonist or partial agonist activity may be used to treat, or alleviate, osteoporosis, including primary osteoporosis such as senile, postmenopausal and juvenile osteoporosis, as well as secondary osteoporosis, such as osteoporosis due to hyperthyroidism or Cushing syndrome (due to corticosteroid treatment), acromegaly, hypogonadism, dysosteogenesis and hypophosphatasemia. Other bone related indications amendable to treat from androgen agonists include osteoporotic fracture, childhood idiopathic bone loss, alveolar bone loss, mandibular bone loss, bone fracture, osteotomy, periodontitis, or prosthetic ingrowth.

Those compounds acting as agonists, or partial agonists, can also be used to stimulate muscle mass in patients afflicted with wasting diseases, such as AIDS, cancer cachexia, burns, renal disease, etc. Patients suffering from trauma, bedsores, age, etc. can also benefits from the anabolic effects of androgens.

### Co-Administration

In a further embodiment of the invention, the compounds of Formula I can be co-administered with other compounds to further enhance their activity, or to minimize potential side effects. For example, potassium channel openers, such as minoxidil, are known to stimulate hair growth and to induce anagen. Examples of other potassium channel openers include (3S,4R)-3,4-dihydro-4-(2,3-dihydro-2-methyl-3-oxopyridazin-6-yl)oxy-3-hydroxy-6-(3-hydroxyphenyl)sulphonyl-2,2,3-trimethyl-2H-benzo[b]pyran, diaxozide, and P1075 which is under development by Leo Pharmaceuticals. Such compounds can be co-administered with the compounds of Formula I to alleviate alopecia.

Thyroid hormone is also known to stimulate hair growth. Synthetic thyroid hormone replacements (i.e., thyromimetics) have also been shown to stimulate hair growth. Such thyromimetics have been described in the literature previously. The reader's attention is directed to European Patent Application No. 1262177, the contents of which are hereby incorporated by reference, for a discussion of such compounds and their use to alleviate alopecia. One particular compound of interest is 2-{4-[3-(4-Fluoro-benzyl)-4-hydroxy-phenoxy]-3,5-dimethyl-phenyl}-2H-[1,2,4]triazine-3,5-dione. Such compounds can be co-administered with the compounds of Formula I to alleviate alopecia.

Anti-androgens can work by a number of different mechanisms. For example, some compounds block the conversion of testosterone to 5-α-dihydrotestosterone, which is responsible for the biological effect in many tissues. 5-Alpha-reductase inhibitors, such as finasteride, have been shown to stimulate hair growth and to decrease sebum production. Finasteride is commercially available from Merck under the trade name Propecia®. Examples of other 5-α-reductase inhibitors include dutasteride (Glaxo Smithkline). Such compounds can be co-administered with the compounds of Formula I to alleviate alopecia and/or to decrease sebum production.

Protein kinase C inhibitors have also been shown to stimulate hair growth and induce anagen. Calphostin C, which is a selective inhibitor of protein kinase-C, has been shown to induce anagen. Other selective protein kinase C inhibitors, such as hexadecylphosphocholine, palmitoyl-DL-carnitine chloride, and polymyxin B sulfate have also been shown to induce anagen. [Skin Pharmacol Appl Skin Physiol 2000 May-August; 13(3-4):133-42]. Any such protein kinase C inhibitor can be co-administered with a compound of Formula I to alleviate alopecia.

Immunophilins are a family of cytoplasmic proteins. Their ligands include cyclosporin and FK506. They are derived from fungi and were developed primarily for their potent immunosuppressive properties. Cyclosporin binds to the proteins, cyclophilins, while FK506 binds to FK binding proteins (FKBPs). All of these compounds have been shown to stimulate hair growth and induce anagen. Any such immunophilin ligands can be co-administered with a compound of Formula I to alleviate alopecia.

Acyl CoA cholesterol acyl transferase (ACAT) inhibitors were initially evaluated for the treatment of elevated serum cholesterol. It was subsequently discovered that these compounds decrease sebum production (U.S. Pat. No. 6,133,326). Any such ACAT inhibitor can be co-administered with a compound of formula I to decrease sebum production, alleviate oily skin, etc.

Antibiotics, such as tetracycline and clindamycin, have been used to alleviate acne. The antibiotic eradicates the microorganism, Propionbacterium acnes, leading to a reduction in the patient's acne. The compounds of Formula I can be co-administered with any antibiotic suitable for the treatment of acne.

Retinoids, such as isotretinoin, have been shown to decrease sebum production and are used to treat acne. These retinoids can be co-administered with a compound of Formula I in order to decrease sebum production and/or to treat acne.

Estrogen and progesterone have each been shown to decrease sebum production. These compounds, or any synthetic agonist of such compounds, may be co-administered with a compound of formula I in order to decrease sebum production.

As used in this application, co-administered refers to administering a compound of Formula I with a second medicinal, typically having a differing mechanism of action, using a dosing regimen that promotes the desired result. This can refer to simultaneous dosing, dosing at different times during a single day, or even dosing on different days. The compounds can be administered separately or can be combined into a single formulation. Techniques for preparing such formulations are described below.

### Formulations

If desired, the compounds can be administered directly without any carrier. However, to ease administration, they will typically be formulated into pharmaceutical carriers. Likewise, they will most typically be formulated into dermatological, or cosmetic carriers. In this application the terms “dermatological carrier” and “cosmetic” carrier are being used interchangeably. They refer to formulations designed for administration directly to the skin or hair.

Pharmaceutical and cosmetic compositions can be manufactured utilizing techniques known in the art. Typically an effective amount of the compound will be admixed with a pharmaceutically/cosmetically acceptable carrier.

For oral administration, the compounds can be formulated into solid or liquid preparations such as capsules, pills, tablets, lozenges, melts, powders, suspensions, or emulsions. Solid unit dosage forms can be capsules of the ordinary gelatin type containing, for example, surfactants, lubricants and inert fillers such as lactose, sucrose, and cornstarch or they can be sustained release preparations.

In another embodiment, the compounds of Formula I can be tableted with conventional tablet bases such as lactose, sucrose, and cornstarch in combination with binders, such as acacia, cornstarch, or gelatin, disintegrating agents such as potato starch or alginic acid, and a lubricant such as stearic acid or magnesium stearate. Liquid preparations are prepared by dissolving the active ingredient in an aqueous or non-aqueous pharmaceutically acceptable solvent, which may also contain suspending agents, sweetening agents, flavoring agents, and preservative agents as are known in the art.

For parenteral administration, the compounds may be dissolved in a physiologically acceptable pharmaceutical carrier and administered as either a solution or a suspension. Illustrative of suitable pharmaceutical carriers are water, saline, dextrose solutions, fructose solutions, ethanol, or oils of animal, vegetative, or synthetic origin. The pharmaceutical carrier may also contain preservatives, buffers, etc., as are known in the art. When the compounds are being administered intrathecally, they may also be dissolved in cerebrospinal fluid as is known in the art.

The compounds of this invention will typically be administered topically. As used herein, topical refers to application of the compounds (and optional carrier) directly to the skin and/or hair. The topical composition according to the present invention can be in the form of solutions, lotions, salves, creams, ointments, liposomes, sprays, gels, foams, roller sticks, or any other formulation routinely used in dermatology.

Thus, a further embodiment relates to cosmetic or pharmaceutical compositions, in particular dermatological compositions, which comprise at least one of the compounds corresponding to Formula I above. Such dermatological compositions will contain from 0.001% to 10% w/w % of the compounds in admixture with a dermatologically acceptable carrier, and more typically, from 0.1 to 5 w/w % of the compounds. Such compositions will typically be applied from 1 to 4 times daily. The reader's attention is directed to Remington's Pharmaceutical Science, Edition 17, Mack Publishing Co., Easton, Pa. for a discussion of how to prepare such formulations.

The compositions according to the invention can also consist of solid preparations constituting cleansing soaps or bars. These compositions are prepared according to the usual methods.

The compounds can also be used for the hair in the form of aqueous, alcoholic or aqueous-alcoholic solutions, or in the form of creams, gels, emulsions or mousses, or alternatively in the form of aerosol compositions also comprising a propellant under pressure. The composition according to the invention can also be a hair care composition, and in particular a shampoo, a hair-setting lotion, a treating lotion, a styling cream or gel, a dye composition, a lotion or gel for preventing hair loss, etc. The amounts of the various constituents in the dermatological compositions according to the invention are those conventionally used in the fields considered.

The medicinal and cosmetics containing the compounds of the invention will typically be packaged for retail distribution (i.e. an article of manufacture). Such articles will be labeled and packaged in a manner to instruct the patient how to use the product. Such instructions will include the condition to be treated, duration of treatment, dosing schedule, etc.

The compounds of Formula I may also be admixed with any inert carrier and utilized in laboratory assays in order to determine the concentration of the compounds within the serum, urine, etc., of the patient as is known in the art. The compounds may also be used as a research tool.

### Use in Livestock

In addition to the therapeutic and cosmetic uses described above, the compounds may also be used to promote the growth of animals, especially livestock. The compounds will increase the rate at which the animals gain weight, increase the leanness of the resulting meat and improve the efficiency of feed utilization. This may be accomplished by administering an effective amount of a compound of Formula I to an animal receiving adequate nutrition to support growth (i.e. sufficient calories, amino acids, vitamins, minerals, essential fats, etc).

To simplify administration, the compound is typically mixed with animal feeds or prepared in the form of an animal-feed premix, concentrate, or supplement which can be blended with animal feeds. Regardless of the procedure selected, the compound will typically be present at levels of from about 0.05 to 500 ppm in the feed.

Animal-feed premixes, supplements or concentrates can be prepared by mixing on a weight basis about 0.5 to 50% of a compound with about 50 to 99.5% of an edible diluent. Diluents suitable for use in the manufacture of animal-feed supplements, concentrates, and premixes include the following: corn meal, soybean meal, bone meal, alfalfa meal, cottonseed oil meal, urea, molasses, and other similar materials. Use of the diluents in feed supplements, concentrates, and premixes improves uniformity of distribution of the active ingredient in the finished feed.

Feeds for swine, cattle, sheep, fish, and goats typically contain about 0.05 to 400 grams of active ingredient per ton of feed. Poultry and domestic-pet feeds range from about 0.05 to 400 grams per ton of feed.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention. The following examples and biological data are being presented in order to further illustrate the invention. This disclosure should not be construed as limiting the invention in any manner.

### EXAMPLES

The General analytical methods used in Examples 1-77, are set forth below, unless specifically stated otherwise:

### 1) Mass Spectroscopy:

MS Conditions: Combi RP3 50×4.6 mm column, 45° C., gradient in 3.5 min, hold 0.5 min

### 2) High Performance Liquid Chromatography:

HPLC conditions: Supelco Discovery C18, 250×4.6 mm, Flow rate=1.5 mL/min,
80/20 to 10/90H₂O+0.1% TFA/ACN+0.1% TFA over 20 min, hold 5 min

### 3) Optical Rotation:

### Conditions: Wavelength: 589 nm, Temp: 24.6° C., Solvent: CHCl₃

### 4) Melting Point:

Determined on a capillary melting point apparatus (either Thomas Hoover or Mel-Temp).

### 5) Liquid Chromatographic Mass Spectroscopy “LCMS”:

Mobile phase: 50-2% H₂O in 3.5 min, hold 0.5 min, run time 4 min; stationary phase: Phenomenex Develosil Combi RP3 50×4.6 mm Column; 45° C. (unless indicated otherwise).

Examples 1-36, 45-56, 72-75 and 79-91 below demonstrate the synthesis of compounds according to the general procedure of Scheme 1 above wherein Structure 1 is 4-fluoro-2-trifluoromethyl-benzonitrile.

Examples 37-42 and 57 below demonstrate the synthesis of compounds according to the general procedure of Scheme 1 above wherein Structure 1 is 3-chloro-4-fluoro-benzonitrile.

Examples 43-44 and 58-71 below demonstrate the synthesis of compounds according to the general procedure of Scheme 1 above wherein Structure 1 is 2-chloro-4-fluoro-benzonitrile.

Examples 71A and 71B below demonstrate the synthesis of compounds according to the general procedure of Scheme 1 above wherein Structure 1 is 4-fluoro-2-methoxy-benzonitrile.

### Example 1

Example 1 illustrates the preparation of a racemic mixture of 4-(1-phenyl-ethoxy)-2-trifluoromethyl-benzonitrile according to Step A of the synthetic route described in Reaction Scheme I. It specifically describes the ether formation (Step A) utilizing 4-fluoro-2-trifluoromethyl-benzenitrile as the structure 1 starting material and sec-phenethyl alcohol as the starting material of Structure 2.

### Example 1

### 4-(1-Phenyl-ethoxy)-2-trifluoromethyl-benzonitrile

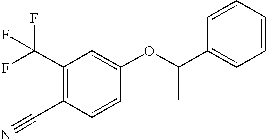

The ether formation was carried out by mixing sec-phenethyl alcohol (1.22 g, 10 mmol), 4-fluoro-2-trifluoromethyl-benzenitrile (1.89 g, 10 mmol), K₂CO₃ (4 g) and DMF (dried, 50 mL) as described in Scheme I. The reaction mixture was, heated to 90° C. for 4 hours. Afterwards, the reaction mixture was cooled to room temperature. The cooled reaction mixture was poured into 100 mL water and extracted with ethyl acetate (EtOAc). The EtOAc solution was washed with water (4 times) and brine (1 time), concentrated and the title product was purified by column chromatography [SiO₂ gel, EtOAc/hexanes (1:1)]. 1.72 g of the desired product was recovered as an oil. (Analysis: C16H12F3NO: calculated C, 65.98; H, 14.15, N, 4.81. found C, 65.84, H, 3.94, N, 4.84), LCMS=>95% pure); MS m/z 2.92 (M⁺H)

### Examples 2 and 3

Examples 2 and 3 illustrate the preparation of the (S) and (R) enantiomers of the compound of Example 1 by utilizing the appropriate enantiomeric form of the alkanol instead of the racemic firm.

### Example 2

### (S)-4-(1-Phenyl-ethoxy)-2-trifluoromethyl-benzonitrile

The (S) enantiomer of the compound of Example 1, (S)-4-(1-phenyl-ethoxy)-2-trifluoromethyl-benzonitrile, was prepared according to the reaction of Example 1 utilizing (S)-(+)-sec-phenethyl alcohol (1.22 g, 10 mmol) instead of the racemic form. 0.24 g of (S)-4-(1-Phenyl-ethoxy)-2-trifluoromethyl-benzonitrile was recovered as an oil. (Analysis: C16H12F3NO: cal C, 65.98; H, 4.15, N, 4.81. found C, 65.46; H, 3.99; N, 4.83), LCMS=>95% pure)

### Example 3

### (R)-4-(1-Phenyl-ethoxy)-2-trifluoromethyl-benzonitrile

The (R) enantiomer of the compound of Example 1, (R)-4-(1-phenyl-ethoxy)-2-trifluoromethyl-benzonitrile, was prepared according to the reaction of Example 1 utilizing (R)-1-phenyl ethanol (0.41 g, 3.3 mmol) as the starting material, structure 2, and reacting it with 4-fluoro-2-trifluoromethyl-benzenitrile (0.63 g, 3.3 mmol), K₂CO₃ (1 g) and DMF (dried, 20 mL). 0.35 g of the (R)-4-(1-phenyl-ethoxy)-2-trifluoromethyl-benzonitrile was recovered as an oil. (Analysis: C16H12F3NO: cal C, 65.98; H, 4.15, N, 4.81. found C, 65.85; H, 3.97; N, 4.84), LCMS=>95% pure.

### Example 4

### 4-[1-(3-Methoxy-phenyl)-ethoxy]-2-t-trifluoromethyl-benzonitrile

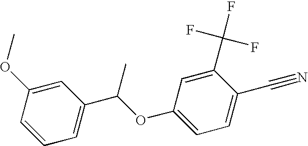

was prepared as described in Example 1, utilizing 1-(3-methoxyl-phenyl)ethanol as the structure 2 starting material. The titled product was recovered as an oil. (Analysis: C17H14F3NO2: cal C, 63.55; H, 4.39, N, 4.36. found C, 63.15; H, 4.19; N, 4.43, LCMS=>95% pure)

### Example 5

### 4-[1-(2-Methoxy-phenyl)-ethoxy]-2-t-trifluoromethyl-benzonitrile

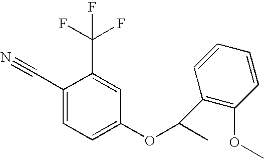

The titled compound was made by a similar method described in Example 1, except that 1-(2-methoxyl-phenyl)ethanol was used as the structure 2 starting material instead of sec-phenethyl alcohol. The titled product was recovered as an oil. (Analysis: C17H14F3NO2: cal C, 63.55; H, 4.39, N, 4.36. found C, 63.79, H, 4.31, N, 4.45, LCMS=>95% pure)

### Example 6

### 4-[(3-Hydroxybenzyl)oxy]-2-(trifluoromethyl)benzonitrile

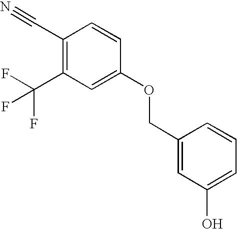

Another method to prepare compounds of the present invention is illustrated in Scheme 2 below.

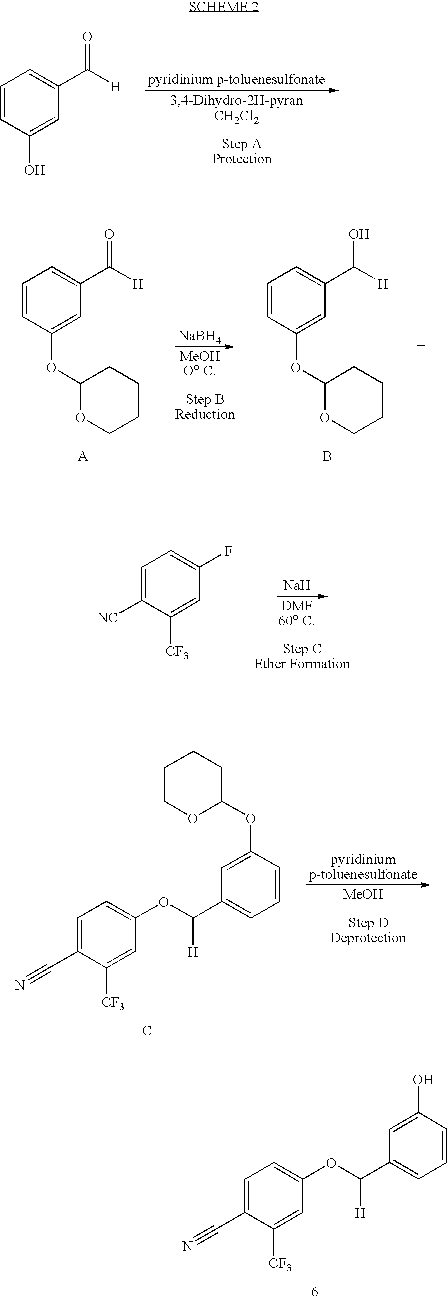

### Step A—Protection

The hydroxyl moiety of 3-hydroxybenzaldehyde was protected by stirring 3-Hydroxybenzaldehyde (10.5 g, 86.0 mmol) and pyridinium p-toluenesulfonate (0.52 g, 2.1 mmol) in methylene chloride (100 mL). The 3,4-dihydropyran (21.7 g, 258 mmol) was then added drop wise by syringe and stirred for two days at room temperature. After two days the reaction was washed with water (500 mL) and condensed. A TLC showed two spots. A silica column was run using 9:1 hexane:ethyl acetate (Hex:EtOAc). 14.69 g (83% yield) of Compound A as a clear yellow oil was obtained.

### Step B—Reduction

The aldehyde, Compound A, was reduced to the alcohol by cooling Compound A (10.0 g, 48.5 mmol) in 100 mL of methanol to 0° C. Sodium borohydride (2.11 g, 55.8 mmol) was then added and the reaction was allowed to stir at 0° C. for twenty minutes. Water was then added and the methanol was removed. The compound was then extracted into ethyl acetate (60 mL, 3 times). The organic layer was then washed with saturated sodium bicarbonate (250 mL), and brine (250 mL). The organic layer was then dried and condensed. The resulting product B (10.1 g, 100% yield) was used as the structure 2 alcohol in Scheme 1 as described in Step C below.

### Step C—Ether Formation

Step C demonstrates the formation of ether utilizing compound B prepared above as the structure 2 of Scheme 1. Compound B (5.00 g, 24 mmol), 4-fluoro-2-(trifluoromethyl)benzonitrile (4.54 g, 24 mmol), and DME (100 mL) were placed in a 300 mL three necked, round bottomed flask equipped with a nitrogen line, condenser, and thermometer. The reaction was cooled to 0° C. Sodium hydride (1.06 g, 26.41 mmol) was then added. The reaction was heated to 60° C. overnight. The reaction was allowed to cool and then water (100 mL) was added. The titled product was extracted into ethyl acetate (100 mL) three times. The organic layers were combined and washed with saturated aqueous sodium bicarbonate (250 mL), and brine (250 mL). The organic layer was then dried and condensed to yield crude product. The crude product was chromatographed using 10:1 Hex:EtOAc to yield compound C (8.55 g, 94% yield).

### Step D—Deprotection

Compound C (8.0 g, 21 mmol) prepared above was deprotected by dissolving it in methanol, adding pyridinium p-toluenesulfonate (0.13 g, 0.53 mmol) and allowing the reaction to stir overnight at room temperature under nitrogen. Aqueous sodium carbonate was then added to the reaction and some solid precipitated. The solid was filtered off and the filtrate was extracted into ethyl acetate (200 mL). The organic layer was dried and condensed to yield desired compound 6 (98.9% pure by LC/MS). M−1=292.2 1H NMR (400 MHz, CHLOROFORM-D) δ ppm 4.78 (s, 1H) 5.12 (s, 2H) 6.83 (m, 1H) 6.89 (m, 1H) 6.96 (ddd, J=7.63, 1.52, 0.85 Hz, 1H) 7.15 (dd, J=8.54, 2.44 Hz, 1H) 7.29 (m, 1H) 7.34 (d, J=2.68 Hz, 1H) 7.74 (m, 1H).

This compound was also prepared by combinatorial chemistry as described on Table I as Example 6A.

### Example 7

### 4-{1-[3-(Tetrahydro-2H-pyran-2-yloxy)phenyl]ethoxy}-2-(trifluoromethyl)benzonitrile

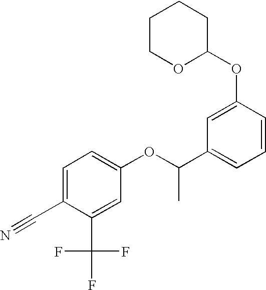

The compound of Example 7 was prepared according to Steps A through C of Scheme 2 in Example 6, using 3′-hydroxy acetophenone as the starting material. The product, 4-{1-[3-(tetrahydro-2H-pyran-2-yloxy)phenyl]ethoxy}-2-(trifluoromethyl)benzonitrile was 97% pure by LC/MS M−1=391.2.

### Example 8

### 4-[1-(3-Hydroxyphenyl)ethoxy]-2-(trifluoromethyl)benzonitrile

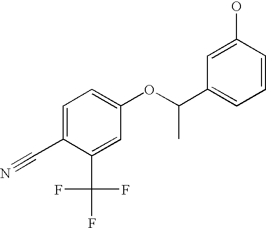

The compound of Example 8 was prepared according to Steps A through C of Scheme 2 for Example 6, using 3-hydroxy acetophenone as the starting material. The product, 4-[1-(3-hydroxyphenyl)ethoxy]-2-(trifluoromethyl)benzonitrile was 98.8% pure by LC/MS. M−1=306.1.

### Examples 9 and 10

Examples 9 and 10 demonstrate the separation of the racemic mixture of Example 8 into its (+) and (−) enantiomers.

The compound of Example 8, 4-[1-(3-hydroxyphenyl)ethoxy]-2-(trifluoromethyl)benzonitrile, was separated into (+) and (−) enantiomers using a SFC chiralcel AD-H 9:1 CO₂:MeOH with a flow rate of 70 mL/min. Retention time (−) 5.2 min, (+) 5.9 min.

### Example 9

### (−)-4-[1-(3-Hydroxyphenyl)ethoxy]-2-(trifluoromethyl)benzonitrile

### Example 10

### (+)-4-[1-(3-Hydroxyphenyl)ethoxy]-2-(trifluoromethyl)benzonitrile

### Example 11

### 4-[1-(3-Hydroxyphenyl)propoxy]-2-(trifluoromethyl)benzonitrile

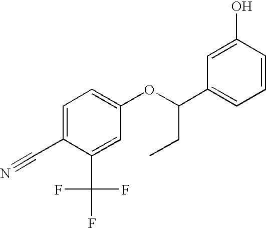

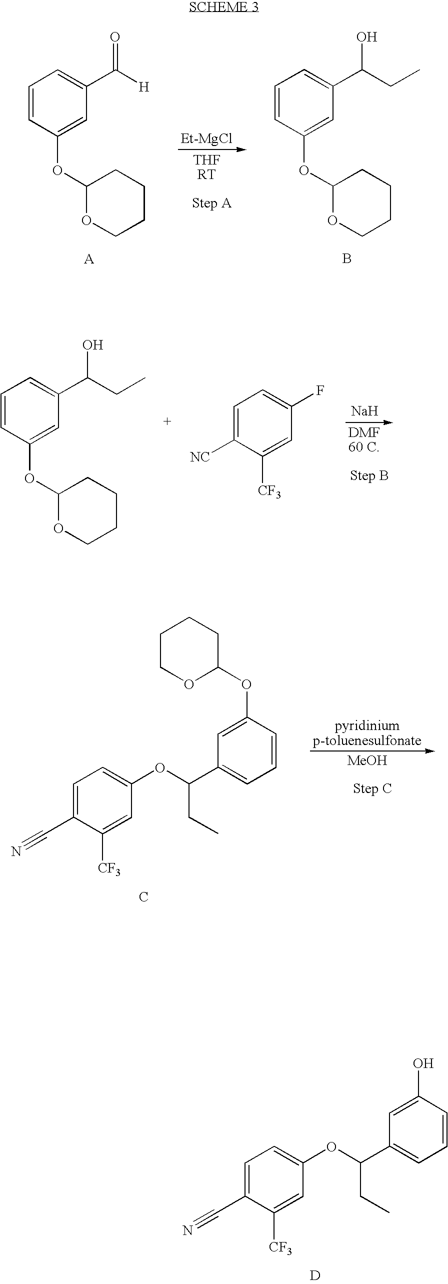

Compound D 4-[1-(3-hydroxyphenyl)propoxy]-2-(trifluoromethyl)benzonitrile was prepared according to Scheme 3 as follows:

### Step A—Nucleophilic Addition to Aldehyde

Compound A (1.25 g, 6.06 mmol) prepared as described in Scheme 2, Step A of Example 6 was placed in a vial under nitrogen. Anhydrous THF (10 mL) was then syringed into the vial. The compound was then transferred to a round bottom flask and cooled to 0° C. The ethyl magnesium chloride (3.79 mL, 2M soln.) was then added drop wise via syringe. The reaction was allowed to warm to room temperature overnight. The reaction was then cooled to 0° C. and aqueous ammonium chloride was added until the pH was 8. The compound was then extracted into EtOAc (100 mL, 4 times). The reaction mixture was dried, condensed and then run on a column using 5:1 Hex:EtOAC. The desired fractions were collected yielding compound B (0.97 g, 68% yield).

### Step B—Ether Formation

Compound B (0.30 g, 1.27 mmol), 4-fluoro-2-(trifluoromethyl)benzonitrile (0.264 g, 1.4 mmol), and DMF (20 mL) were placed in a 100 mL three neck round bottom flask equipped with a nitrogen line, condenser, and thermometer. The reaction was cooled to 0° C. Sodium hydride (0.056 g, 1.4 mmol) was then added. The reaction was heated to 60° C. overnight. The reaction was allowed to cool and then water (50 mL) was added. The product was extracted into ethyl acetate (50 mL, 3×). The organic layers were combined and washed with saturated aqueous sodium bicarbonate (100 mL), and brine (100 mL). The organic layer was then dried and condensed to yield crude product. The crude product was chromatographed using 10:1 Hex:EtOAc. This yielded the desired product C (0.33 g, 64% yield)

### Step C—Deprotection

Compound C (0.33 g, 0.81 mmol) was placed in methanol (10 mL), pyridinium p-toluenesulfonate (0.005 g, 0.20 mmol) was then added and the reaction was allowed to stir overnight at room temperature under nitrogen. Aqueous sodium carbonate (30 mL) was then added to the reaction and some solid precipitated. The solid was filtered off and then the filtrate was extracted into ethyl acetate (50 mL). The organic layer was then dried and condensed to yield crude product. A column was run first flushing through 300 mL hexanes and then 2000 mL of 17% EtOAc was passed through the column. The desired fractions were collected and condensed to yield the desired product 4-[1-(3-hydroxyphenyl)propoxy]-2-(trifluoromethyl)benzonitrile. 99.5% pure by CHN CHN calc. C, 63.55%, H, 4.39%, N, 4.36%. found C, 63.21%, H, 4.39%, N, 4.12%.

### Examples 12 and 13

Examples 12 and 13 demonstrate the separation of the racemic product of Example 11 into (+) and (−) enantiomers utilizing a chiralcel OD column using 9:1 Hex:EPA at a flow rate of 0.8 mL/min. Retention time (+)12.783 min, (−)15.567 min.

### Example 12

### (+)4-[1-(3-Hydroxyphenyl)propoxy]-2-(trifluoromethyl)benzonitrile

### Example 13

### (−)4-[1-(3-Hydroxyphenyl)propoxy]-2-(trifluoromethyl)benzonitrile

### Example 14

### 4-[1-(3-Hydroxyphenyl)butoxy]-2-(trifluoromethyl)benzonitrile

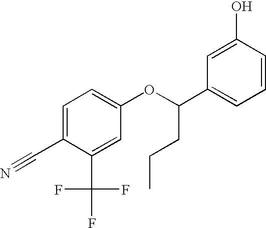

Example 14 was prepared by the method described in Scheme 3 for Example 11, using propyl magnesium chloride in step A instead of EtMgCl. The product, 4-[1-(3-hydroxyphenyl)butoxy]-2-(trifluoromethyl)benzonitrile, was 99.5% pure by CUN. Calc. C, 64.47%, H, 4.81% N, 4.18%. found C, 64.25% H, 4.79% N, 4.17%.

### Example 15

### (+)4-[1-(3-Hydroxyphenyl)butoxy]-2-(trifluoromethyl)benzonitrile

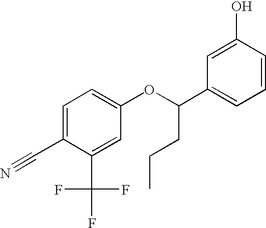

The racemic product of Example 14 was separated into (+) and (−) enantiomers on a chiralcel OD column using 9:1 Hex:IPA with a flow rate of 0.8 mL/min to yield the (+) and (−) enantiomers. Retention time (−) 11.169 min, (+) 13.402 min.

### Example 16

### 4-{[1-(3-Hydroxyphenyl)prop-2-enyl]oxy}-2-(trifluoromethyl)benzonitrile

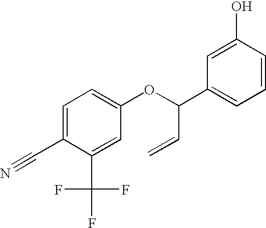

Example 16 was prepared by the method described in Scheme 3 for Example 11, using vinyl magnesium chloride in step A instead of Et-MgCl. The product 4-{[1-(3-hydroxyphenyl)prop-2-enyl]oxy}-2-(trifluoromethyl)benzonitrile was 99.5% pure by CHN. Calc. C, 63.95%, H, 3.79%, N, 4.39%. found C, 64.23%, H, 3.91%, N, 4.10%.

### Example 17

### 4-{[1-(3-Hydroxyphenyl)but-3-enyl]oxy}-2-(trifluoromethyl)benzonitrile

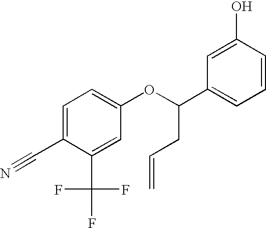

Example 17 was prepared by the method described in Scheme 3 for Example 11, using allyl magnesium chloride in step A instead of Et-MgCl. The product 4-{[1-(3-hydroxyphenyl)but-3-enyl]oxy}-2-(trifluoromethyl)benzonitrile was 99.5% pure by CHN. Calc. C, 64.86%, H, 4.23%, N. 4.20%. found C, 64.51%, H, 4.37%, N, 4.09%.

### Examples 18 and 19

The racemic product of Example 17 was separated into (+) and (−) enantiomers on a chiralcel AD column using 9.5:0.5 Hex:IPA with a flow rate of 70 mL/min. Retention time (+) 5 min, (−) 13 min.

### Example 18

### (+)4-{[1-(3-Hydroxyphenyl)but-3-enyl]oxy}-2-(trifluoromethyl)benzonitrile

### Example 19

### (−)4-{[1-(3-Hydroxyphenyl)but-3-enyl]oxy}-2-(trifluoromethyl)benzonitrile

### Example 20

### 4-[1-(3-Hydroxyphenyl)-3-methylbutoxy]-2-(trifluoromethyl)benzonitrile

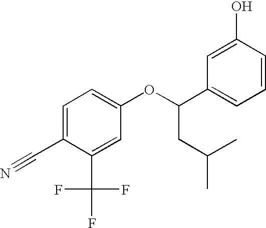

Example 20 was prepared by the method described in Scheme 3 for Example 11, using isobutyl magnesium chloride in step A. 99.6% pure by LCMS. M−1=348.1.

### Example 21

### (+)4-[1-(3-Hydroxyphenyl)-3-methylbutoxy]-2-(trifluoromethyl)benzonitrile

The racemic product of Example 20 was separated into the (+) and (−) enantiomers on a Chiralcel AS-H column using 8.5:1.5 CO₂:MeOH with a flow rate of 4 mL/min to yield. Retention time (+) 1.8 min., (−) 2.1 min. The compound of Example 21 is the (+) enantiomer.

### Example 22

### 4-[1-(2-Chloro-phenyl)-ethoxy]-2-trifluoromethyl-benzonitrile

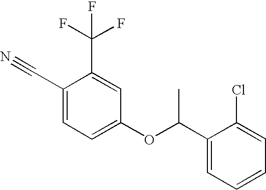

1-(2-Chloro-phenyl)-ethanol (0.82 g, 5.29 mmol) was dissolved in anhydrous tetrahydrofuran (25 mL) and purged with dry nitrogen. Sodium hydride (60% in mineral oil, 0.22 g, 5.55 mmol) was added. After 10 minutes at ambient temperature, 4-fluoro-2-trifluoromethyl-benzonitrile (1.0 g, 5.3 mmol) was added in one portion. The reaction was stirred for 2 hours at ambient temperature before partitioning between ethyl acetate and water. The organic layer was washed with water and brine, dried over anhydrous sodium sulfate, evaporated to dryness, and chromatographed on a 40-g Isco Redisep® silica gel column using a gradient of 5 to 50% ethyl acetate in hexanes to provide 1.03 g (59.8%) of the title compound. HPLC 100%. MS m/z 324 (M−H)⁻.

### Example 23

### 4-[1-(4-Chloro-phenyl)-ethoxy]-2-trifluoromethyl-benzonitrile

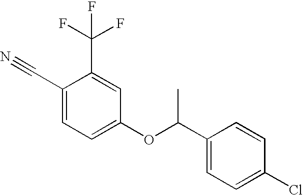

The title compound was prepared in a manner analogous to Example 22 utilizing 1-(4-chloro-phenyl)-ethanol as the starting alcohol and obtaining the title product in 30.1% yield. HPLC>98%. MS itz/z 324 (M−H)⁻.

### Example 24

### 4-[1-(2-Fluoro-phenyl)-ethoxy]-2-trifluoromethyl-benzonitrile

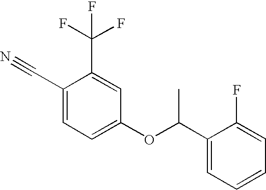

The title compound was prepared in a manner analogous to Example 22 using 1-(2-fluoro-phenyl)-ethanol as the starting alcohol. The title compound was obtained in 37.3% yield. HPLC>98%. MS m/z 308 (M−H)⁻.

### Example 25

### 4-[1-(4-Fluoro-phenyl)-ethoxy]-2-trifluoromethyl-benzonitrile

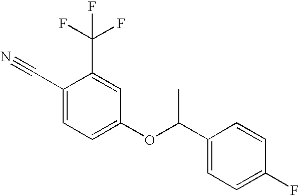

The title compound was prepared in a manner analogous to Example 22 using 1-(4-fluoro-phenyl)-ethanol as the starting alcohol. The title compound was obtained in 39.1% yield. HPLC>99%. MS m/z 308 (M−H)⁻.

### Example 26

### 4-[1-(3-Chloro-phenyl)-ethoxy]-2-trifluoromethyl-benzonitrile

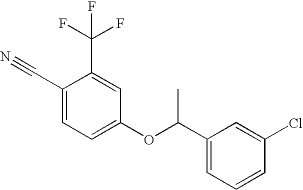

The title compound was prepared in a manner analogous to Example 22 using 1-(3-chloro-phenyl)-ethanol as the starting alcohol. The title compound was obtained in 30.1% yield. HPLC>98%. MS ni/z 324 (M−H)⁻.

### Example 27

### 4-(1-o-Tolyl-ethoxy)-2-trifluoromethyl-benzonitrile

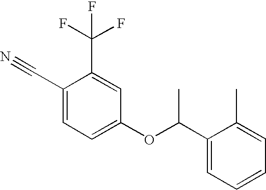

The title compound was prepared in a manner analogous to Example 22 using 1-O— tolyl-ethanol as the starting alcohol. The title compound was obtained in 85.5% yield. HPLC>98%. MS nm/z 304 (M−H)⁻.

### Example 28

### 4-(1-m-Tolyl-ethoxy)-2-trifluoromethyl-benzonitrile

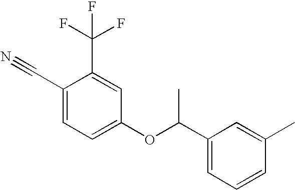

The title compound was prepared in a manner analogous to Example 22 using 1-m-tolyl-ethanol as the starting alcohol. The title compound was obtained in 54.5% yield. HPLC>98.0%. MS m/z 304 (M−H)⁻.

### Example 29

### (±)-4-[1-(4-Cyano-3-trifluoromethyl-phenoxy)-ethyl]-benzoic Acid Methyl Ester

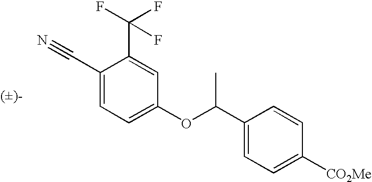

(±)-4-[1-(4-cyano-3-trifluoromethyl-phenoxy)-ethyl]-benzoic acid methyl ester was prepared as follows:

To a cooled solution (0° C.) of methyl-4-(1-hydroxyethyl)benzoate (1.00 g, 5.549 mmol) and 4-fluoro-2-(trifluoromethyl)benzonitrile (1.049 g, 5.549 mmol) in anhydrous dimethylformamide (8 mL) was added NaH (0.222 g as a 60% dispersion in mineral oil). The reaction mixture was allowed to warm to room temperature and was then stirred under nitrogen for 16 hr. The crude reaction mixture was added to ethyl acetate (150 ml) and was washed with saturated aqueous ammonium chloride (2×150 mL), water (1×150 mL), and saturated aqueous sodium chloride (1×150 mL). The organic layer was dried over anhydrous magnesium sulfate, filtered and concentrated. The crude material was chromatographed using hexanes and ethyl acetate (4:1) to afford 0.800 g (41.27% yield) of a viscous colorless oil; ¹H NMR (400 MHz; CDCl₃) δ 8.03 (d, 2H, J=8.3 Hz), 7.63 (d, 1H, J=8.3 Hz), 7.40 (d, 2H, J=8.3 Hz), 7.26 (apparent s occluded by solvent, 1H), 6.97 (dd, 1H, J=8.54, 2.44 Hz), 5.43 (q, 1H, J=6.34 Hz), 3.90 (s, 3H), 1.69 (d, 3H, 6.34 Hz); MS (APCI+) 373.1 ([M+1]+Na); CHN theoretical/actual: C, 61.89/61.90, H, 4.04/4.02, N, 4.01/3.94, F 16.32/16.20.

### Example 30

### 4-[1-(3-Cyano-phenyl)-ethoxy]-2-trifluoromethyl-benzonitrile

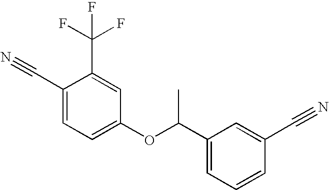

The title compound was prepared in a manner analogous to Example 22 using 3-(1-hydroxy-ethyl)-benzonitrile prepared as below to achieve a 78.6% yield of the title compound. HPLC 100.0%. MS m/z 315 (M−H)⁻.

### 3-(1-Hydroxy-ethyl)-benzonitrile

2-Acetylbenzonitrile (1.0 g, 6.89 mmol) was dissolved in anhydrous methanol (20 mL), treated with sodium borohydride (0.52 g, 13.8 mmol) and stirred at ambient temperature for 18 hours. A saturated solution of ammonium chloride was added and the mixture was extracted with ethyl acetate. The combined organics were washed with water and brine, dried over anhydrous sodium sulfate, evaporated and chromatographed using a gradient of 50 to 100% ethyl acetate in hexanes to provide 0.95 g (93.7%) of 3-(1-Hydroxy-ethyl)-benzonitrile. MS m/z/z 148 (M+W)⁺.

### Example 31

### 2-Trifluoromethyl-4-[1-(2-trifluoromethyl-phenyl)-ethoxy]-benzonitrile

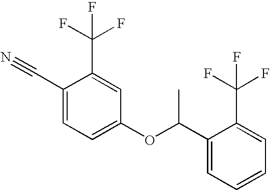

The title compound was prepared in a manner analogous to Example 22 using 1-(2-trifluoromethyl-phenyl)-ethanol as the starting alcohol. The title compound was obtained in 41.9% yield. HPLC 98.9%. MS ni/z 358 (M−H)⁻.

### Example 32

### 2-Trifluoromethyl-4-[1-(3-trifluoromethyl-phenyl)-ethoxy]-benzonitrile

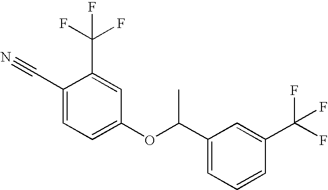

The title compound was prepared in a manner analogous to Example 22 using 1-(3-trifluoromethyl-phenyl)-ethanol as the starting alcohol. The title compound was obtained in 41.9% yield. HPLC 98.1%. MS m/z 358 (M−H)⁻.

### Example 33

### 4-(1-Pyridin-3-yl-ethoxy)-2-trifluoromethyl-benzonitrile

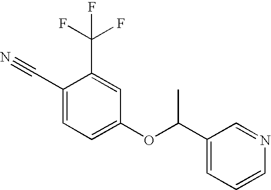

The title compound was prepared in a manner analogous to Example 22 using 1-pyridin-3-yl-ethanol as the starting alcohol. The title compound was obtained in 84.1% yield. HPLC>98%. MS m/z 291 (M−H)⁻.

### Example 34

### 4-(1-Pyridin-3-yl-ethoxy)-2-trifluoromethyl-benzonitrile

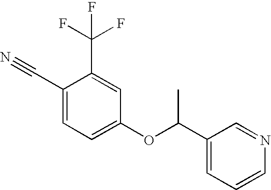

The racemic compound prepared in Example 33, 15.4 g was purified by chiral HPLC using a Chiralcel OD column eluting with 20% isopropanol in hexanes to provide 7.6 g of the desired enantiomer. Chiral HPLC 100% (100% ee (enantioenriched). HPLC 100%. MS m/z 291 (M−H)⁻.

### Example 35

### 4-(1-Pyridin-2-yl-ethoxy)-2-trifluoromethyl-benzonitrile

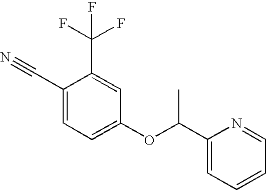

The title compound was prepared in a manner analogous to Example 22 using 1-pyridin-2-yl-ethanol prepared as shown below as the starting alcohol to achieve a 93.2% yield. HPLC>99.0%. MS m/z 291 (M−H)⁻.

### 1-Pyridin-2-yl-ethanol

2-Acetylpyridine (1.0 g, 8.26 mmol) was dissolved in anhydrous methanol (20 mL) and treated with sodium borohydride (0.62 g, 16.51 mmol) and stirred at ambient temperature 18 hours. A saturated solution of ammonium chloride was added and the mixture was extracted with ethyl acetate. The combined organics were washed with water and brine, dried over anhydrous sodium sulfate, evaporated and chromatographed using a gradient of 50 to 100% ethyl acetate in hexanes to provide 0.57 g (56.1%) of the title compound. MS m/z 124 (M+H)⁺.

### Example 36

### (R)-(+)-4-(1-Pyridin-2-yl-ethoxy)-2-trifluoromethyl-benzonitrile

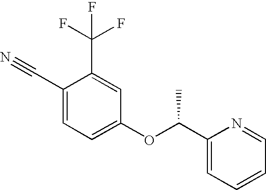

(R)-(+)-4-(1-Pyridin-2-yl-ethoxy)-2-trifluoromethyl-benzonitrile as prepared according to the procedure of Example 35 with the exception that the starting alcohol was (R)-2-(1-Hydroxyethyl)pyridine (3.58 g, 29.1 mmol). After chromatography using a gradient of 0 to 50% ethyl acetate in hexanes 4.35 g (56.5%) of the title compound was recovered. HPLC 100%. MS m/z 291 (M−H)⁻.

An alternate method to prepare the title compound was used to purify the racemic compound prepared in Example 35 by chiral HPLC using a Chiralcel OD column eluting with 20% isopropanol in hexanes to provide 88.8 mg of the desired enantiomer with a retention time of (+) 13.491 min; (−) 11.390 min. Chiral HPLC 99.96 HPLC 96.3%. MS m/z 291 (M⁻H)⁻.

### Example 37

### 3-Chloro-4-(1-methyl-1-phenyl-ethoxy)-benzonitrile

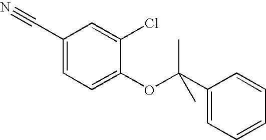

2-Phenyl-2-propanol (0.191 g, 14.0 mmol) was dissolved in 10 mL DMF and cooled to 0° C. NaH (60% in oil, 0.062 g, 15.0 mmol) was added and the mixture stirred for 10 min. Then 3-chloro-4-fluorobenzonitrile (0.200 g, 13.0 mmol) was added and the reaction stirred over a weekend. The reaction mixture was poured into 100 mL ice water and stirred vigorously. The solid precipitate was filtered off and suction dried to give 0.106 g off-white solid. (mp 60-62° C., LCMS=91% pure).

### Example 38

### (R)-3-Chloro-4-(1-phenyl-ethoxy)-benzonitrile

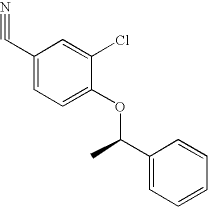

(R)-3-chloro-4-(1-phenyl-ethoxy)-benzonitrile was prepared as described for Example 37 with the exception that the starting alcohol was R-(+)-1-phenyl-ethanol. The crude reaction mixture in DMF was pour into 100 mL of ice water, and extracted three times with ethyl acetate. The combined organic layers were washed with water, then twice with brine, dried over magnesium sulfate, filtered and concentrated rotovapped to give a colorless oil. LCMS purity=69%. CHN calc. C, 69.91% H, 4.69% N, 5.43%. found C, 69.02% H, 5.11% N, 5.25% water 0.40%.

### Example 39

### (S)-3-Chloro-4-(1-phenyl-ethoxy)-benzonitrile

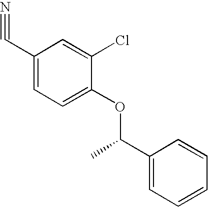

(S)-3-Chloro-4-(1-phenyl-ethoxy)-benzonitrile was prepared as described in Example 38, with the exception that the starting material was S-(−)-1-phenyl-ethanol instead of R-(+)-1-phenyl-ethanol. LCMS purity 93%. CHN calc. C, 69.91% H, 4.69% N, 5.43%. found C, 70.05% H, 5.07% N, 5.04% water 0.40%.

### Example 40

### 3-Chloro-4-(3-methyl-benzyloxy)-benzonitrile

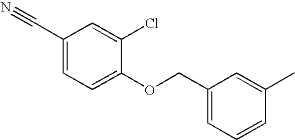

3-Chloro-4-(3-methyl-benzyloxy)-benzonitrile was prepared as described in Example 37 using 3-methylbenzyl alcohol as the starting material. The title product was recovered as a white solid (mp=73-75° C., LCMS=100% pure, M-=256).

### Example 41

### 3-Chloro-4-(3-hydroxy-benzyloxy)-benzonitrile

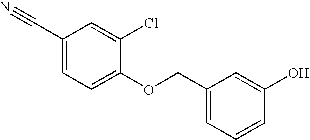

3-Chloro-4-(3-hydroxy-benzyloxy)-benzonitrile was prepared as in Example 1, starting with 3-triisopropylsilanoxy-benzyl alcohol and 3-chloro-4-fluoro-benzonitrile as the starting reactants. The product of the coupling reaction (2.62 mmol) was dissolved in 10 mL THF, then 3.9 mL of tetrabutylammonium fluoride (1.0 M in THF), and 0.15 mL acetic acid were added and stirred overnight at room temperature. The reaction mixture was poured into 100 mL of ice water, then extracted three times with ethyl acetate. The combined organic layers were extracted twice with water then once with brine, dried over magnesium sulfate, filtered and rotoevaporated to give a crude yellow oil. This oil was chromatographed on silica gel with 20% ethyl acetate in chloroform to give 0.1466 g of a colorless oil. (Analysis: CHN calc. C, 64.75% H, 3.88% N, 5.39%. found C, 63.62% H, 3.40% N, 5.07% water 0.64%). LCMS purity=84%.

### Example 42

### 3-Chloro-4-(1-methyl-1,2-diphenyl-ethoxy)-benzonitrile

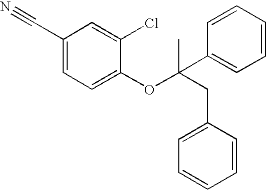

3-Chloro-4-(1-methyl-1,2-diphenyl-ethoxy)-benzonitrile was prepared as described in Example 41 using 1-methyl-1,2-diphenyl-ethanol and 3-chloro-4-fluoro-benzonitrile as the starting reactants. The product was a yellow oil. LCMS purity 80%.

### Example 43

### 2-Chloro-4-(cyclopropyl-phenyl-methoxy)-benzonitrile

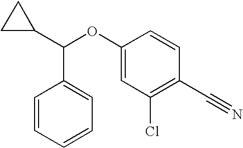

To a solution of 24 mg of cyclopropyl-phenyl-methanol in 150 uL of THF at room temperature, 150 uL of a 1.0 M solution of tert-butoxide in THF was added. This mixture was stirred for 15 minutes, then transferred via syringe to a solution of 23 mg of 2-chloro-4-fluorobenzonitrile in 150 uL of THF. The reaction was shaken at room temperature for 16 h, concentrated, diluted with 250 uL of DMF, filtered and then purified by reverse phase chromatography (Shimadzu semi prep HPLC) to give 9 mg (20%) of the title compound as a colorless oil. GC/MS MIZ 283 (calc 283.1).

### Example 44

### 2-Chloro-4-(1-phenyl-propoxy)-benzonitrile)

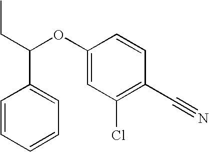

To a solution of 21 mg of 1-phenyl-propan-1-ol in 150 uL of THF at room temperature, 150 uL of a 1.0 M solution of tert-butoxide in THF was added. This mixture was stirred for 15 minutes, then transferred via syringe to a solution of 23 mg of 2-chloro-4-fluorobenzonitrile in 150 uL of THF. The reaction was shaken at room temperature for 16 h, concentrated, diluted with 250 uL of DMF, filtered and then purified by reverse phase chromatography (Shimadzu semi prep HPLC) to give 7 mg (17% of the title compound) as a colorless oil. GC/MS M/Z 271 (calc 271.1).

### Examples 45-71

The compounds of Examples 45-71 were prepared by combinatorial chemistry, using the general synthetic method of Reaction Scheme 1.

Reactant 1 was either 4-fluoro-2-(trifluoromethyl)-benzonitrile, 4-fluoro-2-(chloro)-benzonitrile, or 4-fluoro-3-(chloro)-benzonitrile. The other reactant was an appropriate alcohol as described by structure 2. A variety of combinatorial methods were used. The specifics of each are described below. The letter identifying each method is used in the examples below to explain how the compounds were made, purified, characterized.

### Combinatorial Methods

### I) Synthetic Methods

### Method A:

To 0.33 mL of a 0° C. 1M solution of the corresponding aryl fluoride in tetrahydrofuran “THF” (0.3 mmol) was added 0.6 mL of a 1 M solution of potassium t-butoxide in THF (0.6 mmol) and 0.3 mL of a 1 M solution of the corresponding alcohol (0.3 mmol) in THF. The resultant mixtures were shaken and allowed to warm to room temperature over approximately 18 hours. The solvent was removed in vacuo using a Genevac HT-12 to obtain a sample that was then purified by reverse phase HPLC.

### Method B:

To 1 mL of a 0° C. 0.3M solution of the corresponding aryl fluoride in tetrahydrofuran “THF” (0.3 mmol) was added 0.6 mL of a 1 M solution of potassium t-butoxide in THF (0.6 mmol) and 0.3 mL of a 1 M solution of the corresponding alcohol (0.3 mmol) in THF. The resultant mixtures were shaken and allowed to warm to room temperature over approximately 72 hours. The solvent was removed in vacuo using a Genevac HT-12 to obtain a sample that was then purified by reverse phase HPLC.

### Method C:

To 1 mL of a 0.3M solution of the corresponding aryl fluoride in tetrahydrofuran “THF” (0.3 mmol) was added a 1 mL slurry of a 0.63 M solution of sodium hydride (60%) in THF (0.63 mmol) and 0.3 mL of a 11.0M solution of the corresponding alcohol (0.3 mmol) in THF. The resultant mixtures were shaken at room temperature over approximately 18 hours. The reactions were quenched with methanol and macroporous tosic acid resin (0.32 mmol, loading 1.53 mmol/g). The resultant mixture was shaken at room temperature for approximately 18 hours. Filtered the reaction, rinsing with THF. The solvent was removed in vacuo using a Genevac HT-12 to obtain a sample that was then purified by reverse phase HPLC.

### Method D:

To 1 mL of a 0.25M solution of the corresponding aryl fluoride in N,N′-dimethylformamide “DMF” (0.25 mmol) was added a 1 mL slurry of a 0.25 M solution of sodium hydride (60%) in THF (0.25 mmol) and 0.25 mL of a 1 M solution of the corresponding alcohol (0.25 mmol) in THF. The resultant mixtures were shaken at room temperature over approximately 18 hours. The reactions were quenched with methanol and macroporous tosic acid resin (0.3 mmol, loading 1.53 mmol/g). The resultant mixture was shaken at room temperature for approximately 18 hours. Filtered the reaction, rinsing with THF. The solvent was removed in vacuo using a Genevac HT-12 to obtain a sample that was then purified by reverse phase HPLC.

### Method E:

To 1 mL of a 0.3M solution of the corresponding aryl fluoride in DMF (0.3 mmol) was added a 1 mL slurry of a 0.6 M solution of sodium hydride (60%) in DMF (0.6 mmol) and 0.33 mL of a 1 M solution of the corresponding alcohol (0.33 mmol) in THF. The resultant mixtures were shaken at room temperature over approximately 18 hours. The reactions were quenched with methanol and macroporous tosic acid resin (0.61 mmol, loading 4.07 mmol/g). The resultant mixture was shaken at room temperature for approximately 18 hours. Filtered the reaction, rinsing with methanol. The solvent was removed in vacuo using a Genevac HT-12 to obtain a sample that was then purified by reverse phase HPLC.

### Method F:

To a solution of the corresponding aryl fluoride (0.2 mmol) and the corresponding alcohol (0.200 mmol) in DMF (1 mL) is added 0.5 mL of 0.6 M slurry of sodium hydride (60%) in DMF (0.3 mmol). The resultant mixtures were shaken at room temperature for 48 hours. The reactions were quenched with water (0.5 mL). The solvent is evaporated in vacuo. To the concentrated reaction mixtures is added methylene chloride (3 mL) and water (2 mL). The organic layer is filtered through silica (0.5 g) solid phase extraction column and evaporated to yield material that was purified by reverse phase HPLC.

### II) HPLC Methods (High Performance Liquid Chromatography)

### Method A:

Column: BHK 30×100 mm ODS-O/B 5 mm C-18.
Flow rate: 30 mL/min
Solvent: A=Acetonitrile w/3% 1-Propanol; B=Water w/3% 1-Propanol
Method: 0-6 min: 100% B; 6-10 min: 100% A

### Method B:

Column: BHK 30×100 mm ODS-O/B 5 mm C-18.
Flow rate: 30 mL/min
Solvent: A=Acetonitrile w/3% 1-Propanol; B=Water w/3% 1-Propanol
Method: 0-7 min: 100% A; 7-10.5 min: 100% B

### Method C:

Column: YMC 30×100 mm ODS-A 5 mm C-18.
Flow rate: 30 mL/min
Solvent: A=Acetonitrile w/3% 1-Propanol; B=Water w/3% 1-Propanol
Method: 0-7 min: 10% A, 90% B; 7-10 min: 100% A

### Method D:

Column: YMC 30×100 mm ODS-A 5 mm C-18.
Flow rate: 30 mL/min
Solvent: A=Acetonitrile w/3% 1-Propanol; B=Water w/3% 1-Propanol
Method: 0-6 min: 10% A, 90% B; 6-10.5 min: 100% A

### Method E:

Column: BHK 30×100 mm ODS-OB 5 mm C-18.
Flow rate: 30 mL/min
Solvent: A=Acetonitrile w/3% 1-Propanol; B=Water w/3% 1-Propanol
Method: 0-6.5 min: 15% A, 85% B; 6.5-10.5 min: 100% A

### Method F:

Column: YMC 30×100 mm ODS-A 5 mm C-18.
Flow rate: 30 mL/min
Solvent: A=Acetonitrile w/3% 1-Propanol; B=Water w/3% 1-Propanol
Method: 0-6.5 min: 10% A, 90% B; 6.5-10.5 min: 100% A

### Method G:

Column: Xterra 30×100 mm ODS-A 5 mm C-18.
Flow rate: 30 mL/min
Solvent: A=Acetonitrile w/3% 1-Propanol; B=Water w/3% 1-Propanol
Method: 0-7.5 min: 15% A, 85% B; 7.5-10.5 min: 100% A

### Method H

Column: Sunfire 19×100 mm Prep C18 5 micron
Flow Rate: 30 mL/1 min
Solvent: A=acetonitrile w/0.1% formic acid; B=water w/0.1% formic acid
Method: 0-1 min: 25% A; 1-7.5 min: 25% B to 100% B

### III) LCMS (Liquid Chromatography Mass Spectrum) Methods

### Method A:

LCMS: Lunc Phenyl Hexyl 50 mm×4.6 mm, 3 mm column (Solvent: A=Water w/10 mM Ammonium Acetate; B=Acetonitrile w/0.005M Formic Acid, Method: 0-2 min: 80% A, 20% B; 2-4.1 min: 2% A, 98% B; 4.1-6 min: 80% A, 20% B

### Method B:

LCMS: YMC ODS-AQ 50 mm×4.6 mm, 3 mm column (Solvent: A=Water w/10 mM Ammonium Acetate; B=Acetonitrile w/0.005M Formic Acid, Method: 0-3 min: 90% A, 10% B; 3-5.1 min: 2% A, 98% B; 5.1-7 min: 90% A, 10% B

### Method C:

LCMS: YMC Pack Pro C18, 50 mm×4.6 mm, 3 mm column (Solvent: A=Water w/0.1M Formic Acid; B=Acetonitrile w/0.1M Formic Acid, Method: 0-1.5 min: 95% A, 5% B; 1.5-4.1 min: 2% A, 98% B; 4.1-7 min: 95% A, 5% B.

### Method D:

LCMS: YMC ODS-AQ, 50 mm×4.6 mm, 3 mm column (Solvent: A=Water w/0.1M Formic Acid; B=Acetonitrile w/0.1M Formic Acid, Method: 0-2.5 min: 80% A, 20% B; 2.5-5.1 min: 2% A, 98% B; 5.1-7 min: 80% A, 20% B.

### Method E:

LCMS: Atlantis C18, 50 mm×4.6 mm, 3 mm column (Solvent: A=Water w/0.1M Formic Acid; B=Acetonitrile w/0.1M Formic Acid, Method: 0-3 min: 85% A, 15% B; 3-5.1 min: 2% A, 98% B; 5.1-7 min: 85% A, 15% B.

### Method F:

LCMS: Atlantis C18, 50 mm×4.6 mm, 3 mm column (Solvent: A=Water w/0.1M Formic Acid; B=Acetonitrile w/0.1M Formic Acid, Method: 0-2.5 min: 80% A, 20% B; 2.5-5.1 min: 2% A, 98% B; 5.1-7 min: 80% A, 20% B.

### Method G:

LCMS: Alltech Alltima C18, 150 mm×3.2 mm, 5 mm column (Solvent: A=Water w/0.1M Formic Acid; B=Acetonitrile w/0.1M Formic Acid, Method: 0-6 min: 65% A, 35% B; 6-8.1 min: 2% A, 98% B; 8.1-10 min: 65% A, 35% B.

Compounds made by the combinatorial methods described above are demonstrated in Table I below. Ret. Time=retention time in minutes.

TABLE I

| Example | | | | | | |
|---|---|---|---|---|---|---|
| # | Structure | Name | Synthesis | HPLC | LCMS | LCMS Properties |
| 45 | 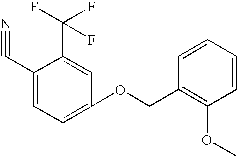 | 4-(2-Methoxy- benzyloxy)-2- trifluoromethyl- benzonitrile | B | B | B | MS: 308.12 (M + 1 for C16H12F3NO2) Ret. Time: 3.86, Purity: 100 |
| 46 | 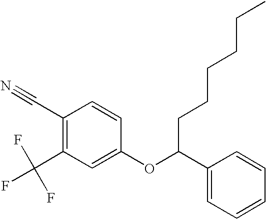 | 4-(1-Phenyl- heptyloxy)-2- trifluoromethyl- benzonitrile | B | C | C | MS: 362.23 (M + 1 for C21H22F3NO) Ret. Time: 3.47 Purity: 100 |
| 47 | 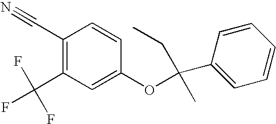 | 4-(1-Methyl-1- phenyl-propoxy)-2- trifluoromethyl- benzonitrile | B | C | C | MS: 320.19 (M + 1 for C₁₈H₁₆F₃NO) Ret. Time: 2.98 Purity: 100 |
| 48 | 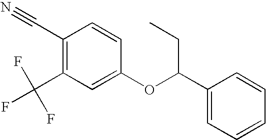 | 4-(1-Phenyl- propoxy)-2- trifluoromethyl- benzonitrile | B | C | C | MS: 306.17 (M + 1 for C₁₇H₁₄F₃NO) Ret. Time: 2.88 Purity: 100 |
| 50 | 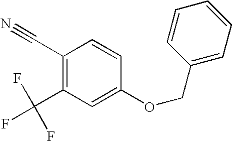 | 4-Benzyloxy-2- trifluoromethyl- benzonitrile | B | B | B | MS: 278.1 (M + 1 for C₁₅H₁₀F₃NO) Ret. Time: 3.83 Purity: 100 |
| 51 | 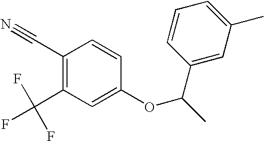 | 4-(1-m-Tolyl- ethoxy)-2- trifluoromethyl- benzonitrile | B | C | C | MS: 306.2 (M + 1 for C₁₇H₁₄F₃NO) Ret. Time: 2.87 Purity: 100 |
| 52 | 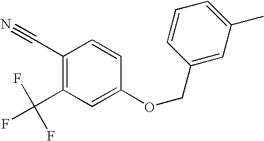 | 4-(3-Methyl- benzyloxy)-2- trifluoromethyl- benzonitrile | C | D | D | MS: 292.14 (M + 1 for C₁₆H₁₂F₃NO) Ret. Time: 3.54 Purity: 100 |
| 53 | 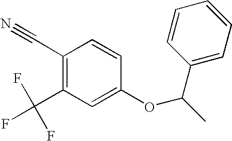 | 4-(1-Phenyl-ethoxy)- 2-trifluoromethyl- benzonitrile | B | C | C | MS: 292.17 (M + 1 for C₁₆H₁₂F₃NO) Ret. Time: 2.77 Purity: 100 |
| 6A | 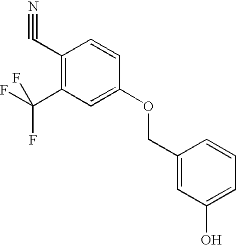 | 4-(3-Hydroxy- benzyloxy)-2- trifluoromethyl- benzonitrile | D | F | F | MS: 294.2 (M + 1 for C₁₅H₁₀F₃NO₂) Ret. Time: 3.32 Purity: 100 |
| 54 | 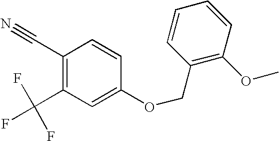 | 4-(2-Methoxy- benzyloxy)-2- trifluoromethyl- benzonitrile | B | B | B | MS: 308.12 (M + 1 for C₁₆H₁₂F₃NO₂) Ret. Time: 3.86 Purity: 100 |
| 55 | 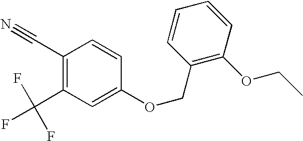 | 4-(2-Ethoxy- benzyloxy)-2- trifluoromethyl- benzonitrile | C | D | D | MS: 322.17 (M + 1 for C₁₇H₁₄F₃NO₂) Ret. Time: 3.56 Purity: 100 |
| 56 | 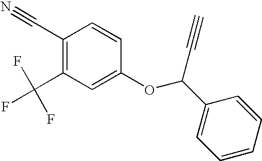 | 4-(1-Phenyl-prop-2- ynyloxy)-2- trifluoromethyl- benzonitrile | B | C | C | MS: 300.16/301.05 doublet (M − 1 for C₁₇H₁₀F₃NO) Ret. Time: 2.52 Purity: 100 |
| 57 | 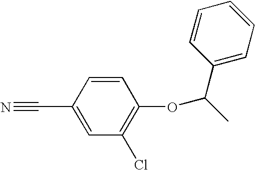 | 3-Chloro-4-(1- phenyl-ethoxy)- benzonitrile | E | G | G | MS: 258.18 (M + 1 for C₁₅H₁₂ClNO) Ret. Time: 5.75 Purity: 100 |
| 58 | 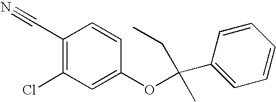 | 2-Chloro-4-(1- methyl-1-phenyl- propoxy)- benzonitrile | B | C | C | MS: 286.33 (M + 1 for C₁₇H₁₆ClNO) Ret. Time: 3 Purity: 100 |
| 59 | 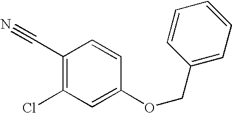 | 4-Benzyloxy-2- chloro-benzonitrile | B | B | B | MS: 244.06 (M + 1 for C₁₄H₁₀ClNO) Ret. Time: 3.8 Purity: 98.92 |
| 60 | 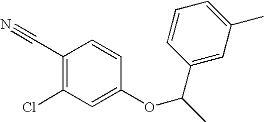 | 2-Chloro-4-(1-m- tolyl-ethoxy)- benzonitrile | B | C | C | MS: 272.26 (M + 1 for C₁₆H₁₄ClNO) Ret. Time: 2.85 Purity: 94.22 |
| 61 | 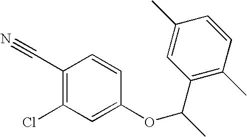 | 2-Chloro-4-[1-(2,5- dimethyl-phenyl)- ethoxy]-benzonitrile | C | D | D | MS: 286.16 (M + 1 for C₁₇H₁₆ClNO) Ret. Time: 3.71 Purity: 90.36 |
| 62 | 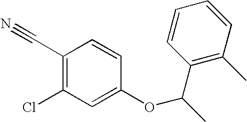 | 2-Chloro-4-[1-(2,6- dimethyl-phenyl)- ethoxy]-benzonitrile | B | C | C | MS: 272.24 (M + 1 for C₁₆H₁₄ClNO) Ret. Time: 2.83 Purity: 91.78 |
| 63 | 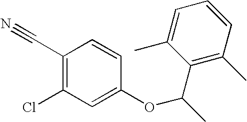 | 2-Chloro-4-[1-(2,6- dimethyl-phenyl)- ethoxy]-benzonitrile | B | C | C | MS: 286.26 (M + 1 for C₁₇H₁₆ClNO) Ret. Time: 2.97 Purity: 100 |
| 64 | 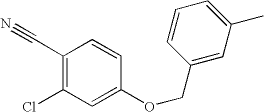 | 2-Chloro-4-(3-methyl-benzyloxy)- benzonitrile | C | D | D | MS: 258.13 (M + 1 for C₁₅H₁₂ClNO) Ret. Time: 3.49 Purity: 100 |
| 65 | 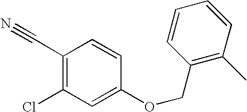 | 2-Chloro-4-(2- methyl-benzyloxy)- benzonitrile | C | D | D | MS: 258.15 (M + 1 for C₁₅H₁₂ClNO) Ret. Time: 3.47 Purity: 100 |
| 66 | 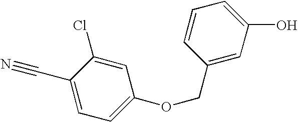 | 2-Chloro-4-(3- hydroxy-benzyloxy)- benzonitrile | D | F | F | MS: 260.14 (M + 1 for C₁₄H₁₀ClNO₂) Ret. Time: 2.86 Purity: 100 |
| 67 | 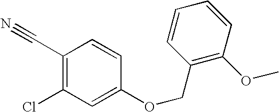 | 2-Chloro-4-(2- methoxy-benzyloxy)- benzonitrile | B | B | B | MS: 272.09 (M − 1 for C₁₅H₁₂ClNO₂) Ret. Time: 3.83 Purity: 98.29 |
| 68 | 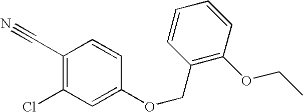 | 2-Chloro-4-(2- ethoxy-benzyloxy)- benzonitrile | C | D | D | MS: 288.11 (M + 1 for C₁₆H₁₄ClNO₂) Ret. Time: 3.57 Purity: 100 |
| 69 | 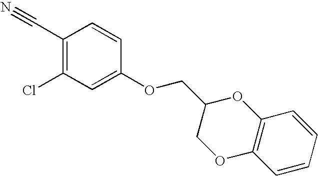 | 2-Chloro-4-(2,3- dihydro- benzo[1,4]dioxin-2- ylmethoxy)- benzonitrile | C | D | D | MS: 302.14 (M + 1 for C₁₆H₁₂ClNO₃) Ret. Time: 3.32 Purity: 100 |
| 70 | 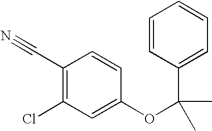 | 2-Chloro-4-(1- methyl-1-phenyl- ethoxy)-benzonitrile | C | D | D | MS: 272.14 (M + 1 for C₁₆H₁₄ClNO) Ret. Time: 3.62 Purity: 99.23 |
| 71 | 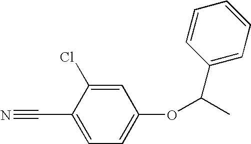 | 2-Chloro-4-(1- phenyl-ethoxy)- benzonitrile | E | G | G | MS: 258.22 (M + 1 for C15H12ClNO) Ret. Time: 5.75 Purity: 100 |
| 71A | 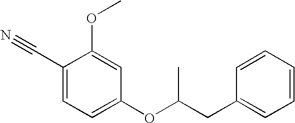 | 2-Methoxy-4-(1- methyl-2-phenyl- ethoxy)-benzonitrile | F | H | F | MS: 268.1 (M + 1 for C₁₇H₁₇NO₂) Ret. Time: 3.52 Purity: 100 |
| 71B | 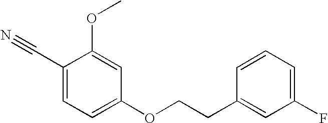 | 4-[2-(3-Fluoro- phenyl)-ethoxy]-2- methoxy-benzonitrile | F | H | F | MS: 272.1 (M + 1 for C₁₆H₁₄FNO₂) Ret. Time: 4.16 Purity: 100 |

### Example 72

### 4-[1-(5-Methoxypyridin-3-yl)ethoxy]-2-(trifluoromethyl)benzonitrile

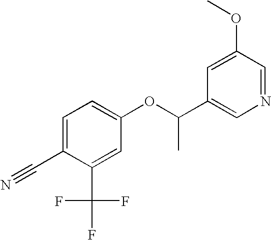

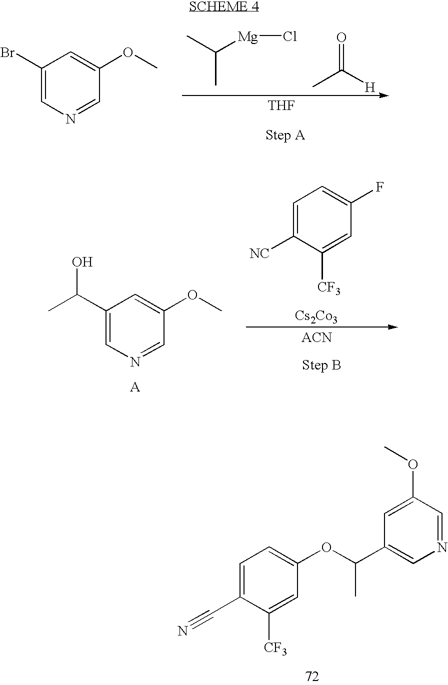

### Step A—Grignard Exchange Reaction.

3-Bromo-5-methoxypyridine (1.00 g, 5.32 mmol) and the isopropyl magnesium chloride (2.19 g, 21.3 mmol) were stirred together for 2 hours in THF (20 mL). A LC/MS was taken showing loss of the bromo. The reaction was then cooled down to −21° C. and the acetylaldehyde (2.34 g, 53.19 mmol) was added. The reaction was allowed to warm to room temperature and stirred overnight. The THF was then removed. Water (100 mL) was added to the reaction and then methylene chloride was used to extract the product (75 mL, 3 times). The methylene chloride layer was washed with brine (75 mL). The methylene chloride layer was dried and condensed to give crude product. The product was placed on a silica column using 2400 mL Hex:EtOAc (5:1) and then 100% EtOAc. The product A (0.503 g, 61.74% yield) was collected and condensed.

Step B—Ether Formation

Compound A (0.503 g, 3.28 mmol), 4-fluoro-2-(trifluoromethyl)benzonitrile (0.68 g, 3.6 mmol) and cesium carbonate (1.2 g, 3.6 mmol) were allowed to stir in acetonitrile (10 mL) for 2 days under nitrogen. The cesium carbonate was then filtered off and washed with ethyl acetate. The reaction mixture was the condensed and chromatographed using a silica column and 3:1 hexane:EtOAc. The desired fractions were condensed to yield compound 72 (0.325, 31% yield) 99.5% pure by CHN calc Carbon 59.63, Hydrogen 4.07, Nitrogen 8.69. Found C, 59.26; H, 4.05; N, 8.57.

### Example 73

### 4-[1-(5-Hydroxypyridin-3-yl)ethoxy]-2-(trifluoromethyl)benzonitrile

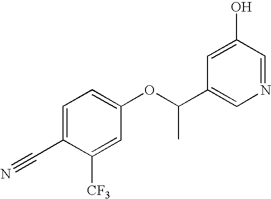

4-[1-(5-Hydroxypyridin-3-yl)ethoxy]-2-(trifluoromethyl)benzonitrile was prepared according to Scheme 5.

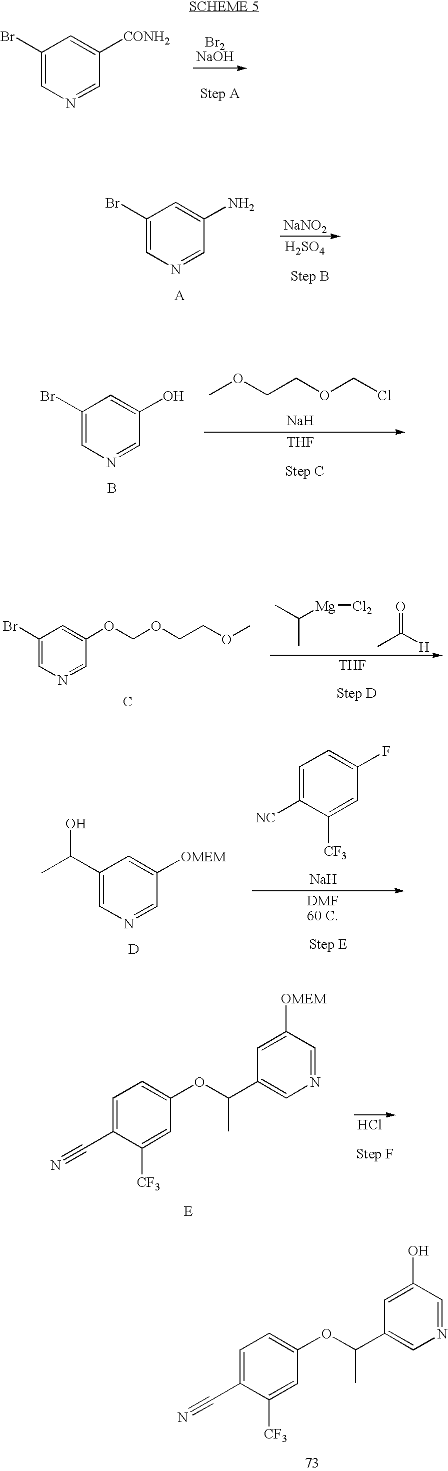

### Step A—Hoffman Rearrangement

Bromine (18.76 g, 117.4 mmol) was added to a solution of sodium hydroxide (23.88 g, 597 mmol) in water (200 mL). To this solution was added 5-bromonicotinamide (20.00 g, 99.49 mmol). The reaction mixture was heated to 75° C. for 45 minutes. The reaction was cooled and acidified with concentrated hydrochloric acid. Some insoluble material was present. The insolubles were removed by filtration. The solution was washed with ethyl acetate (150 mL, 2 times). The aqueous solution was basified with sodium hydroxide solution (pH 10). The mixture was extracted into ethyl acetate (200 mL, 2 times). The ethyl acetate was dried and condensed to yield compound A (10.07 g, 58.5% yield).

### Step B—Diazatization

Compound A (10.00 g, 57.8 mmol) was converted to Compound B (9.46 g, 94% yield) via the procedure outlined in Journal of the American Chemical Society, 1973, 95(22), 7458-7464.

### Step C—Protection

The hydroxyl group of Compound B (0.50 g, 2.87 mmol) was protected as the MEM ether by dissolving in THF (50 mL) and cooling to 0° C. Sodium hydride (0.15 g, 3.74 mmol) was then added and the reaction was allowed to stir for 10 minutes. The MEM chloride (0.57 g, 4.60 mmol) was then added and the reaction was allowed to stir at 0° C. for 5 minutes. The reaction was then allowed to warm to room temperature and stir overnight. Water (150 mL) was then added and the reaction was extracted into EtOAc (100 mL, 3 times). The EtOAc was then washed with saturated NaHCO₃ (100 mL), and brine (100 mL). The EtOAc was dried and condensed to yield compound C (0.68 g, 90.28% yield).

### Step D—Grignard Exchange Reaction

Step D demonstrates the Grignard exchange reaction wherein the pyridyl Grignard reagent is formed by exchange with the isopropyl magnesium chloride followed by the reaction of this Grignard reagent with acetylaldehyde. Compound C (0.49 g, 1.87 mmol) and isopropyl magnesium chloride (0.77 g, 7.5 mmol) were stirred together for 2 hours in THF (20 mL). A LC/MS was taken showing loss of the bromo. The reaction was then cooled down to −21° C. and the acetylaldehyde (0.82 g, 18.7 mmol) was added. The reaction was allowed to warm to room temperature and stirred overnight. The THF was then removed under vacuum. Water (100 mL) was added to the reaction and then methylene chloride was used to extract the product (75 mL, 3 times). The methylene chloride layer was washed with brine (75 mL). The methylene chloride was dried and condensed to give product D (0.43 g, 59% yield), which was used without further purification.

### Step E—Ether Formation

Compound D (3.00 g, 13.20 mmol) and 4-fluoro-2-(trifluoromethyl)benzonitrile (2.75 g, 14.5 mmol) and cesium carbonate (4.7 g, 14.5 mmol) were allowed to stir in acetonitrile (30 mL) for 2 days under nitrogen. The cesium carbonate was then filtered off and washed with ethyl acetate. The reaction mixture was the condensed and chromatographed using a silica column and 1:1 Hexane:Ethyl acetate. The desired fractions were condensed to yield compound E.

### Step F—Deprotection

Compound E (3.41 g, 8.6 mmol) was dissolved in THF (35 mL), methanol (35 mL), and 2N HCl (35 mL). The reaction was allowed to stir at room temp until. LC/MS showed no more starting material. The reaction mixture was then added to an aqueous solution of sodium bicarbonate (100 mL of an 8% solution). The mixture was extracted with ethyl acetate (100 mL, 3 times). The ethyl acetate was dried and condensed to yield crude product. The crude product was placed on a silica column eluting with ethyl acetate to give 73 (2.64 g, 99.5%) 95% pure by LCMS. M+1=309.1.

### Example 74

### (+)4-[1-(5-Hydroxypyridin-3-yl)ethoxy]-2-(trifluoromethyl)benzonitrile

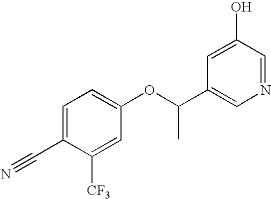

Example 74 was prepared by the method described for Example 73. The racemic product of Example 73 was placed on a chiralpak AS column using 9:1 Hex:EtOH and a flow rate of 10 mL/min to yield the (+) and (−) enantiomers. Retention time: (+)=16.2 min; (−)=12.18 min.

Specific rotation=(+)−59.5 in methanol at 589 nm.

### Example 75

### 4-[2-(4-Cyano-phenyl)-ethoxy]-2-trifluoromethyl-benzonitrile

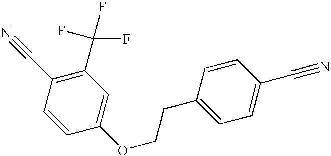

To a solution of 4-fluoro-2-trifluoromethyl-benzonitrile (3 g, 15.9 mmoles) and 4-(2-hydroxy-ethyl)-benzonitrile (2.3 g, 15.9 moles) in 50 mL acetonitrile is added sodium hydride, 60% dispersion in mineral oil (0.890 g, 33.3 mmoles). The reaction mixture is stirred one hour at room temperature. After 1 hour 10 mL water is added to quench the excess sodium hydride. The reaction mixture is dissolved in 150 mL of ethyl acetate and washed with 2 50 mL portions of brine. The organic layer is dried over magnesium sulfate, filtered and evaporated in vacuo. The residue is chromatographed over SiO₂ (gradient 5% to 40% ethyl acetate hexanes) to yield. 4-[2-(4-Cyano-phenyl)-ethoxy]-2-trifluoromethyl-benzonitrile (2.11 g). (Analysis: C₁₇H₁₁F₃N₂O: theory C, 64.56; H, 3.51; N, 8.86. found C, 63.69; H, 3.34; N, 8.67).

### Example 76

### 4-[2-(4-Methoxy-phenyl)-ethoxy]-2-trifluoromethyl-benzonitrile

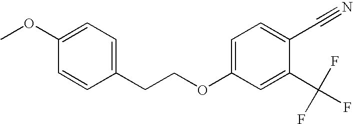

Example 76 was prepared as described in Example 1, with the exception that the starting alcohol was 2-(4-methoxy-phenyl)-ethanol. LCMS purity>99%. CHN calc. CHN calc. C, 63.55%, H, 4.39%, N, 4.36. Found C, 63.44%, H, 4.20%, N, 4.32% (C₁₇H₁₄F₃NO₂).

### Example 77

### 4-[2-(3-Methoxy-phenyl)-ethoxy]-2-trifluoromethyl-benzonitrile

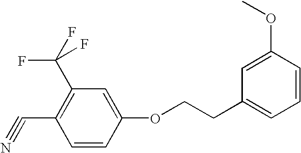

Example 77 was prepared as described in Example 1, with the exception that the starting alcohol, structure 2, was 2-(3-methoxy-phenyl)-ethanol. LCMS purity>99%. CHN calc. C, 63.55%, H, 4.39%, N, 4.36. Found C, 63.41%, H, 4.18%, N, 4.31% (C₁₇H₁₄F₃NO₂)—

### Example 78

### 4-[1-(3-Cyano-phenyl)-ethoxy]-2-trifluoromethyl-benzonitrile

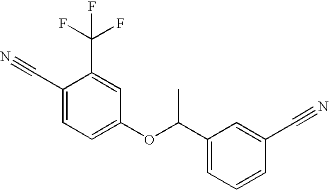

4-[1-(3-Cyano-phenyl)-ethoxy]-2-trifluoromethyl-benzonitrile was prepared by the method of Scheme 1 using 3-(1-hydroxyethyl)benzonitrile (8.81 g, 59.9 mmol), prepared as describe below, as the starting alcohol. After chromatography using a gradient of 0 to 50% ethyl acetate in hexanes, 16.1 g (85%) of the racemic title compound was obtained. A portion of the racemic material was purified by chiral HPLC using a Chiralcel OD column eluting with 20% isopropanol in hexanes to provide 257.6 mg of the desired enantiomer (+) retention time 13.506 min; (−) Rₜ 11.544 min. Chiral HPLC 99.9%. HPLC 98.6%. MS m/z 315 (M⁻H)⁻.

### 3-(1-Hydroxy-ethyl)-benzonitrile

3-Acetylbenzonitrile (12.5 g, 86.3 mmol) was dissolved in anhydrous methanol (100 mL) and treated with sodium borohydride (6.53 g, 172.6 mmol) and stirred at ambient temperature 18 hours. A saturated solution of ammonium chloride was added and the mixture was extracted with ethyl acetate. The combined organics were washed with water and brine, dried over anhydrous sodium sulfate, evaporated, and chromatographed using a gradient of 50 to 100% ethyl acetate in hexanes to provide 8.81 g (69.4%) of the title compound. MS m/z 148 (M+H)⁺.

### Example 79

### 4-(1-Pyridin⁻²-yl-propoxy)-2-trifluoromethyl-benzonitrile

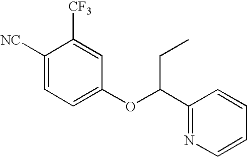

### Step 1—Reduction of Ketone

1-Pyridin-2-yl-propan-1-ol was prepared by the general method of Scheme 2, step B, Example 6, starting with 1-phenyl-propan-1-one and ethanol as solvent. The reaction was stirred at room temperature for approximately 18 hours. The resulting oil was used in Step 2 without further purification.

### Step 2—Ether Formation

The title compound was prepared by the method described in Scheme 4, Example, 72, Step B, except THF was used as solvent and the starting material was 1-pyridin-2-yl-propan-1-ol. The reaction was heated to 60° C. for approximately 18 hours. MS m/z 307.1 (MH+). Elemental analysis: theory C, 62.74; H, 4.28; N, 9.15. Found C, 62.46; H, 4.02; N, 9.05.

### Example 80

### 4-[1-(6-Methoxy-pyridin-2-yl)-ethoxy]-2-trifluoromethyl-benzonitrile

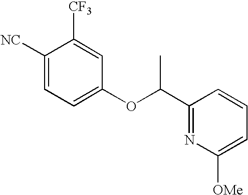

### Step 1—Nucleophilic Addition to Aldehyde

A colorless solution of 2-bromo-6-methoxypyridine (3.06 g, 16.3 mmol) in THF was cooled to −78° C. BuLi (20 mmol, 1.2 equiv) was added over approximately 15 min and the reaction stirred at −78° C. for 1 h: Acetaldehyde (1.1 equiv) was added. The reaction was stirred for 1 h, then it was allowed to warm to room temperature and stirred overnight. The reaction mixture was cooled to 0° C., quenched with water and diluted with EtOAc. The aqueous layer was extracted 3 times with EtOAc. The combined organic layers were dried (MgSO₄) and concentrated. The crude product was purified by CombiFlash flash chromatography with 50% ether/hexane and yielded 1-(6-methoxy-pyridin-2-yl)-ethanol as an orange oil, 1.97 g (79%).

### Step 2—Ether Formation

The title compound was prepared by a method analogous to Example 79 using 1-(6-methoxy-pyridin-2-yl)-ethanol instead of 1-pyridin-2-yl-propan-1-ol. MS niz 323.1 (MH+). Elemental analysis: (C₁₆H₁₃F₃N₂O₂.0.03H₂O): theory C, 59.53; H, 4.08; N, 8.68. Found C, 59.13; H, 3.85; N, 8.55.

### Example 81

### 4-(1-Pyridin-2-yl-butoxy)-2-trifluoromethyl-benzonitrile

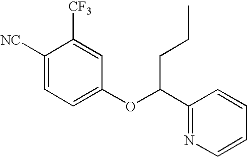

The title compound was prepared by a method analogous to Example 80 using 2-bromopyridine and butyraldehyde as starting materials. MS m/z 321.1 (MH+). HPLC, 94%.

### Example 82

### 4-(2-Phenyl-1-pyridin-2-yl-ethoxy)-2-trifluoromethyl-benzonitrile

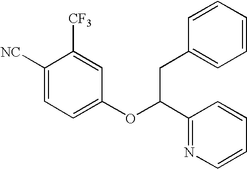

The title compound was prepared by a method analogous to Example 80 using 2-bromopyridine and phenyl-acetaldehyde as starting materials. MS m/z 369.6 (MH+). HPLC, 100%.

### Example 83

### 4-(3-Methyl-1-pyridin-2-yl-butoxy)-2-trifluoromethyl-benzonitrile

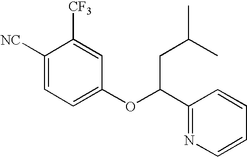

The title compound was prepared by a method analogous to Example 80 using 2-bromopyridine and 3-methyl-butyraldehyde as starting materials. MS m/z 335.2 (MH+). HPLC, 100%.

### Example 84

### 4-(1-Pyridin-2-yl-pentyloxy)-2-trifluoromethyl-benzonitrile

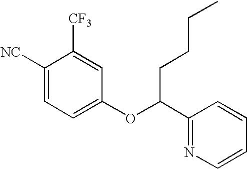

The title compound was prepared by a method analogous to Example 80 using 2-bromopyridine and pentanal as starting materials. MS m/z 335.5 (MH+). Elemental analysis: (C₁₈H₁₇F₃N₂O.0.32H₂O): theory C, 63.57; H, 5.23; N, 8.24; Found C, 63.20; H, 4.93; N, 8.20.

### Example 85

### 4-[3-Methyl-1-(6-methyl-pyridin-2-yl)-butoxy]-2-trifluoromethyl-benzonitrile

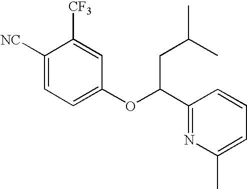

The title compound was prepared by a method analogous to Example 80 using 2-bromo-6-methyl-pyridine and 3-methyl-butyraldehyde as starting materials. MS m/z 349.4 (MH+). HPLC, 95.6%.

### Example 86

### 4-[1-(6-Methyl-pyridin-2-yl)-butoxy]-2-trifluoromethyl-benzonitrile

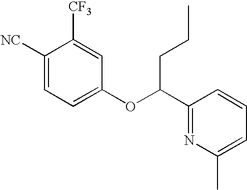

The title compound was prepared by a method analogous to Example 80 using 2-bromo-6-methyl-pyridine and butyraldehyde as starting materials. MS m/z 335.4 (MH+). Elemental analysis: theory C, 64.66; H, 5.13; N, 8.38. Found C, 64.51; H, 5.00; N, 8.26.

### Example 87

### 4-[1-(6-Methyl-pyridin-2-yl)-propoxy]-2-trifluoromethyl-benzonitrile

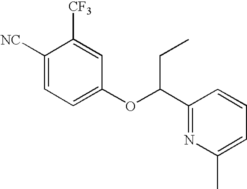

The title compound was prepared by a method analogous to Example 80 using 2-bromo-6-methyl-pyridine and propionaldehyde as starting materials. MS m/z 321.4 (MH+). HPLC, 100%. Elemental analysis: theory C, 63.675; H, 4.72; N, 8.75. Found C, 64.72; H, 4.01; N, 8.53.

### Examples 88-95

For Examples 88 to 95, the analytical LCMS utilized was Phenomenex Luna C18 4.6×150 mm 5 uM, flow rate 1.5 mL/min; gradient 10% to 90% Acetonitrile with 0.1% formic acid/Water with 0.1% formic acid in 8 min; 90% Acetonitrile with 0.1% formic acid/Water with 0.1% formic acid hold for 1.5 minutes.

### Example 88

### 4-(3-Methyl-1-pyridin-3-yl-butoxy)-2-trifluoromethyl-benzonitrile

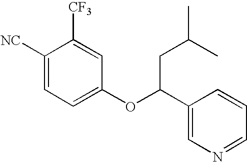

### Step 1—Scheme 4A: Reduction of Ketone

3-Methyl-1-pyridin-3-yl-butan-1-ol was prepared by the general method of Scheme 4, step A, Example 72, starting with 3-bromopyridine (0.5 g, 3.16 mmol) in 20 ml of dry THF was stirred at −20° C. under N₂, then isopropyl magnesium chloride (1.0 g, 13 mmol) was added, the reaction mixture was warmed to 0° C. for 1 hour. The reaction mixture was cooled to −20° C. then isovaleraldehyde (2.7 g, 31 mmol) was added, and the reaction mixture was allowed to warm to room temperature over night. The reaction mixture was concentrated at reduced pressure, extracted with ethyl acetate and washed with saturated ammonium chloride and saturated NaHCO₃. The solution was dried with MgSO₄. The resulting oil was used in Step 2 without further purification. MS m/z 166 (MH+).

### Step 2—Ether Formation

The title compound was prepared by the method described in Scheme 4, step B, Example 72, the starting material was 3-Methyl-1-pyridin-3-yl-butan-1-ol. The reaction was heated to 60° C. for 16 hours; the mixture was then warmed to 80° C. for 24 h before workup. LCMS m/z 335 (MH+) for C₁₈H₁₇F₃N₂O LCMS: RT: 2.95 min. Assay=94.1%.

### Example 89

### 4-[3-Methyl-1-(6-methyl-pyridin-3-yl)-butoxy]-2-trifluoromethyl-benzonitrile

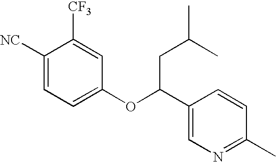

### Step 1—Nucleophilic Addition to Aldehydes

3-Methyl-1-(6-methyl-pyridin-3-yl)-butan-1-ol was prepared by the general method of Example 80, starting with 2-bromo-5-methylpyridine instead of 2-bromo-6-methoxypyridine. The reaction mixture was allowed to warm to room temperature overnight. The reaction was quenched with 1 ml of water. The reaction mixture was concentrated at reduced pressure, extracted with ethyl acetate and washed with saturated NaHCO₃ and brine. The solution was dried with MgSO₄. The crude product was used directly in the next step. MS m/z 180 (MH+).

### Step 2—Ether Formation

The title compound was prepared by a method analogous to Example 79, using 3-Methyl-1-(6-methyl-pyridin-3-yl)-butan-1-ol instead of 1-pyridin-2-yl-propan-1-ol. LCMS m/z 349 (MH+), LCMS: RT: 2.1 min. Assay=87.9%.

### Example 90

### 4-(1-Pyridin-3-yl-propoxy)-2-trifluoromethyl-benzonitrile

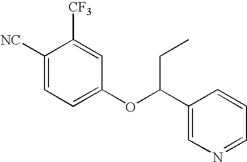

The title compound was prepared by a method analogous to Example 89, using 3-bromopyridine and propionaldehyde as starting materials. LCMS m/z 307 (MH+) LCMS: RT: 2.0 min. Assay=98.8%.

### Example 91

### 4-(2-Phenyl-1-pyridin-3-yl-ethoxy)-2-trifluoromethyl-benzonitrile

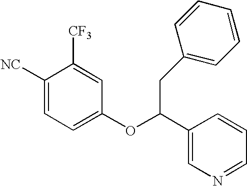

The title compound was prepared by a method analogous to Example 89, using 3-bromopyridine and propionaldehyde as starting materials. LCMS m/z 369 (MH+) LCMS: RT: 2.5 min. Assay=97.2%.

### Example 92

### 4-[1-[5-(2-Methoxy-ethoxymethoxy)-pyridin-3-yl]-propoxy]-2-trifluoromethylenzonitrile

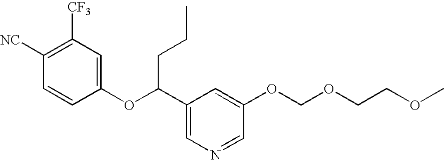

The title compound was prepared by a method analogous to Scheme 4, step B, Example 72, using 1-[5-(2-methoxy-ethoxymethoxy)-pyridin-3-yl]-butan-1-ol and 4-fluoro-2-trifluoromethylbenzonitrile as starting materials. LCMS m/z 425 (MH+) LCMS: RT: 2.9 min. Assay=98.5%.

### Example 93

### 4-{1-[5-(2-Methoxy-ethoxymethoxy)-pyridin-3-yl]-3-methyl-butoxy}-2-trifluoromethyl-benzonitrile

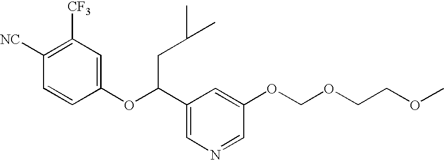

The title compound was prepared by a method analogous to Scheme 4, step B, Example 72, using 1-[5-(2-methoxy-ethoxymethoxy)-pyridin-3-yl]-3-methyl-butan-1-ol and 4-fluoro-2-trifluoromethylbenzonitrile as starting materials. LCMS m/z 439 (MH+) LCMS: RT: 3.2 min. Assay=100%.

### Example 94

### 4-[1-(5-Hydroxy-pyridin-3-yl)-propoxy]-2-trifluoromethyl-benzonitrile

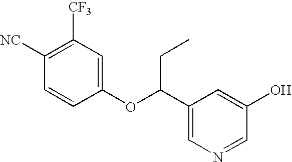

A solution of the crude 4-{1-[5-(2-methoxy-ethoxymethoxy)-pyridin-3-yl]-propoxy}-2-trifluoromethyl-benzonitrile (4.5 g) in 20 ml of 3 N hydrochloric acid, 20 ml of methanol, and 20 ml of THF, the reaction mixtures are heated to 50° C. for 16 hours. The reaction mixture was cooled to room temperature and concentrated at reduced pressure. The reaction mixture was extracted with ethyl acetate and washed with saturated NaHCO₃ and brine. The solution was dried with MgSO₄ and concentrated at reduced pressure. The crude reaction mixture was purified using a gradient of 5% to 55% ethyl acetate/hexane on a BIOTAGE system to give the desired product (2.34 g). LCMS m/z 321 (MH+) LCMS: RT: 1.2 min. Assay=98.8%.

### Example 95

### 4-[1-(5-Hydroxy-pyridin-3-yl)-butoxy]-2-trifluoromethyl-benzonitrile

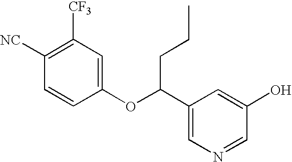

The title compound was prepared by a method analogous to Example 94, using 4-{1-[5-(2-methoxy-ethoxymethoxy)-pyridin-3-yl]-propoxy}-2-trifluoromethyl-benzonitrile and 4-fluoro-2-trifluoromethylbenzonitrile as starting materials. LCMS m/z 337 (MH+) LCMS: RT: 1.5 min. Assay=98.3%.

### Example 96

### AR Binding Assay

The compounds of Formula I have affinity for the androgen receptor. This affinity has been demonstrated for selected compounds using the human receptor. The description below describes how the assay was carried out.

Competitive binding analysis was performed on baculovirus/Sf9 generated hAR extracts in the presence or absence of different concentrations of test agent and a fixed concentration of ³H-dihydrotestosterone (³H-DHT) as tracer. This binding assay method is a modification of a protocol previously described (Liao S., et. al., J. Steroid Biochem., 20:11-17, 1984). Briefly, progressively decreasing concentrations of compounds are incubated in the presence of hAR extract (Chang et al., P.N.A.S., Vol. 89, pp. 5546-5950, 1992), hydroxylapatite, and 1 nM ³H-DHT for one hour at 4° C. Subsequently, the binding reactions are washed three times to completely remove excess unbound ³H-DHT. hAR bound ³H-DHT levels are determined in the presence of compounds (i.e. competitive binding) and compared to levels bound when no competitor is present (i.e. maximum binding). Compound binding affinity to the hAR is expressed as the concentration of compound at which one half of the maximum binding is inhibited. Table II below provides the results that were obtained for selected compounds (reported data is the mean of multiple tests as shown below).

TABLE II

| | | AR | |
|---|---|---|---|
| Example | | Binding | |
| # | | IC₅₀ (nM) | |
| 1 | 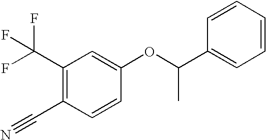 | 10 | (c) |
| 2 | 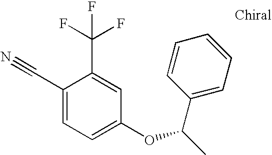 | 13 | (c) |
| 3 | 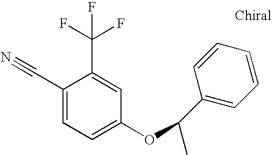 | 3 | (c) |
| 4 | 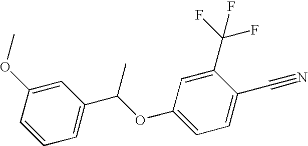 | 78 | (c) |
| 5 | 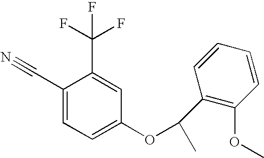 | 312 | (n = 6) |
| 6 | 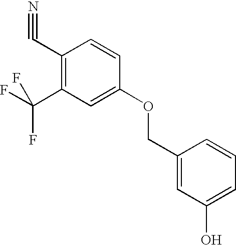 | 12 | (n = 6) |
| 7 | 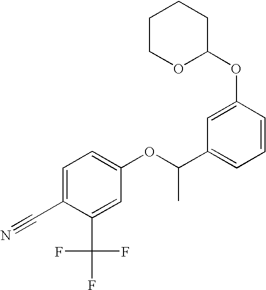 | 383 | (a) |
| 8 | 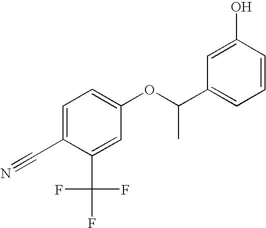 | 21 | (a) |
| 9 | 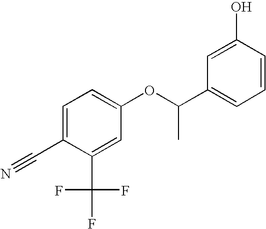 | 56 | (c) |
| 10 | 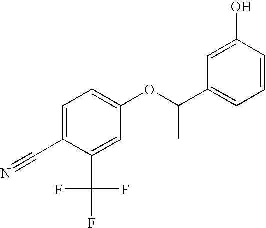 | 3 | (a) |
| 11 | 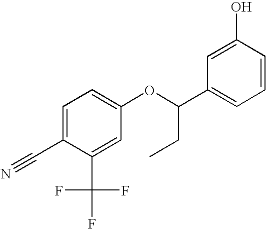 | 11 | (a) |
| 12 | 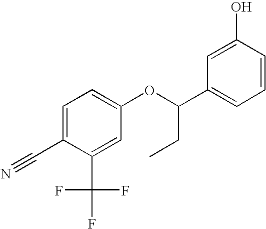 | 19 | (a) |
| 13 | 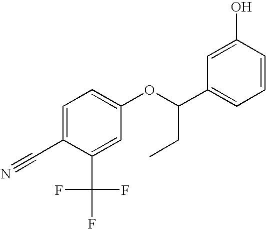 | 23 | (a) |
| 14 | 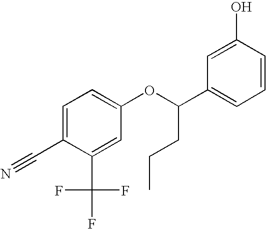 | 18 | (c) |
| 15 | 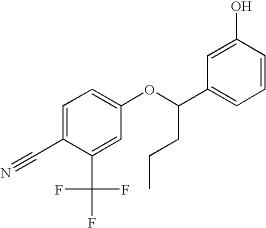 | 20 | (c) |
| 16 | 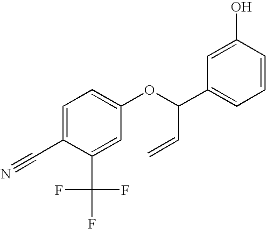 | 11 | (a) |
| 17 | 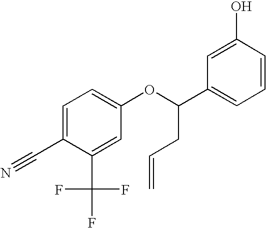 | 14 | (a) |
| 18 | 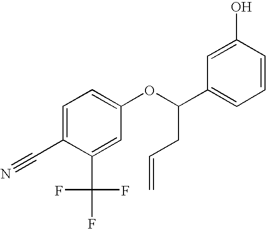 | 17 | (c) |
| 19 | 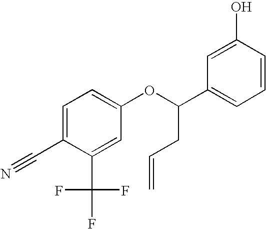 | 185 | (c) |
| 20 | 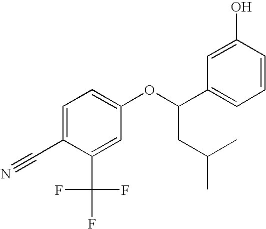 | 183 | (c) |
| 21 | 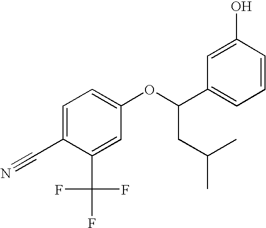 | 66 | (a) |
| 22 | 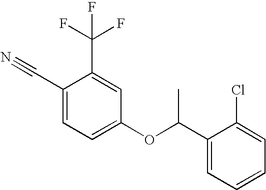 | 108 | (a) |
| 23 | 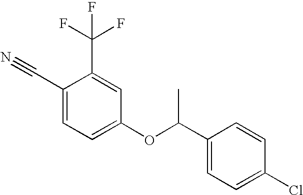 | 307 | (a) |
| 24 | 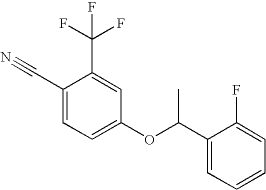 | 79 | (a) |
| 25 | 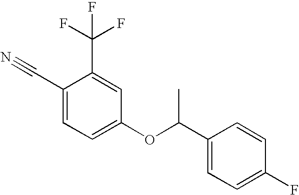 | 137 | (a) |
| 26 | 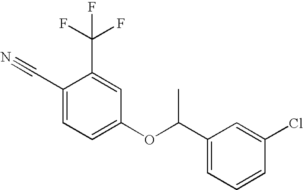 | 122 | (a) |
| 27 | 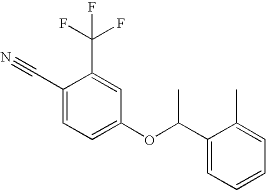 | 439 | (a) |
| 28 | 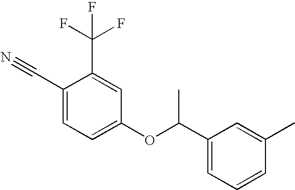 | 50 | (a) |
| 29 | 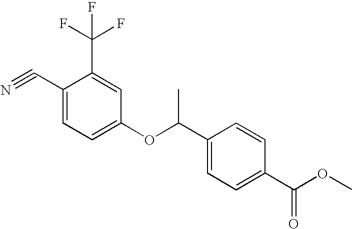 | 102 | (a) |
| 30 | 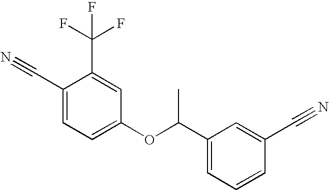 | 22 | (a) |
| 31 | 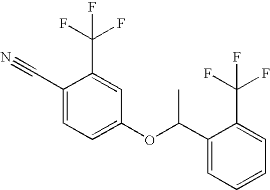 | 146 | (a) |
| 32 | 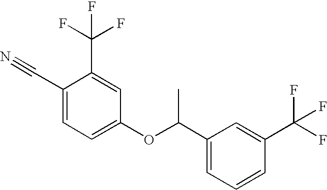 | 214 | (a) |
| 33 | 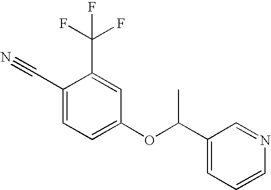 | 192 | (a) |
| 34 | 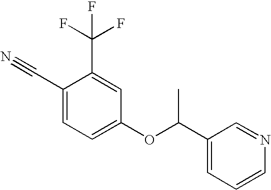 | 141 | (c) |
| 35 | 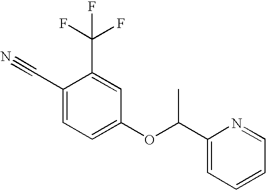 | 180 | (c) |
| 36 | 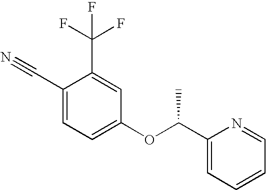 | 54 | (a) |
| 37 | 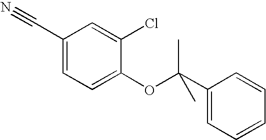 | 184 | (a) |
| 38 | 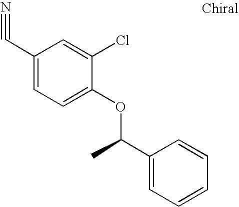 | 281 | (a) |
| 39 | 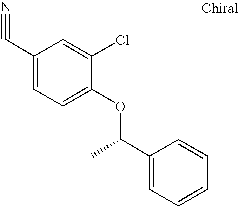 | 410 | (a) |
| 40 | 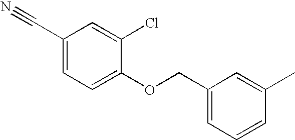 | 783 | (a) |
| 41 | 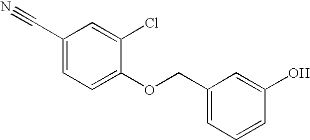 | 76 | (a) |
| 42 | 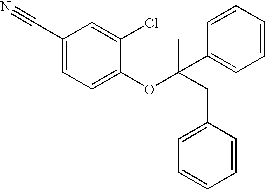 | 377 | (a) |
| 43 | 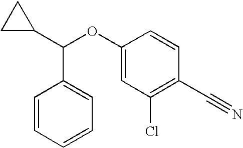 | UA | |
| 44 | 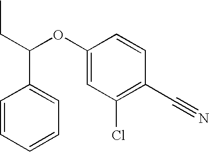 | UA | |
| 45 | 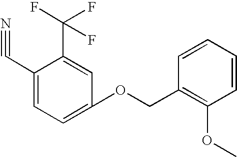 | 38 | (a) |
| 46 | 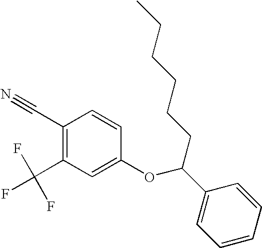 | 204 | (a) |
| 47 | 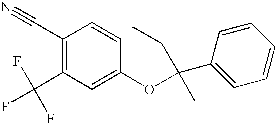 | 461 | (a) |
| 48 | 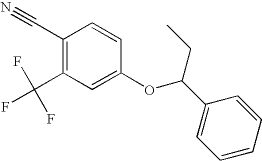 | 38 | (n = 6) |
| 50 | 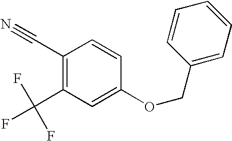 | 174 | (n = 6) |
| 51 | 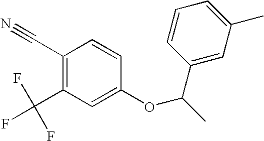 | 108 | (a) |
| 52 | 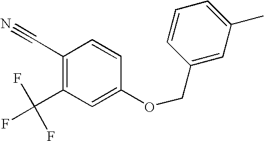 | 302 | (a) |
| 53 | 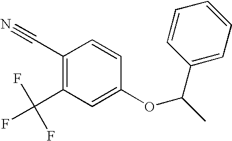 | 8 | (a) |
| 54 | 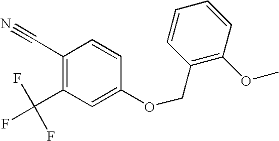 | 331 | (a) |
| 55 | 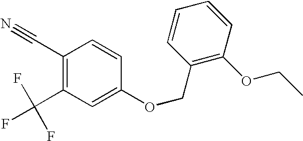 | 125 | (a) |
| 56 | 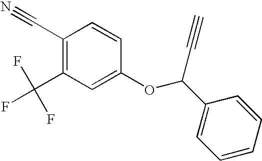 | 74 | (a) |
| 57 | 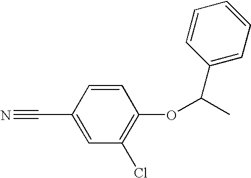 | 240 | (a) |
| 58 | 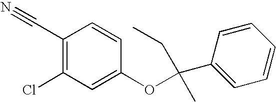 | 162 | (a) |
| 59 | 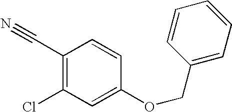 | 38 | (a) |
| 60 | 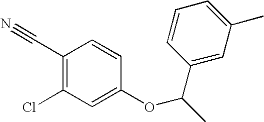 | 74 | (a) |
| 61 | 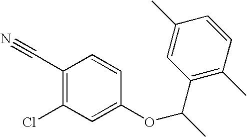 | 61 | (a) |
| 62 | 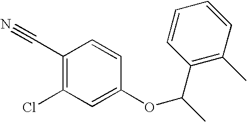 | 52 | (a) |
| 63 | 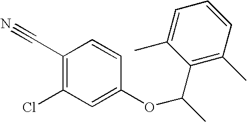 | 328 | (a) |
| 64 | 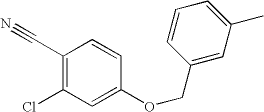 | 74 | (a) |
| 65 | 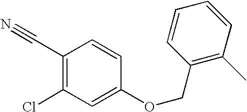 | 129 | (a) |
| 66 | 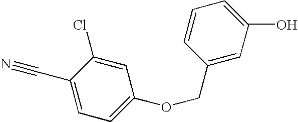 | 11 | (a) |
| 67 | 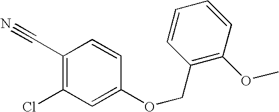 | 99 | (a) |
| 68 | 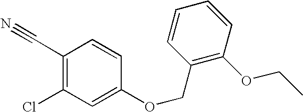 | 213 | (a) |
| 69 | 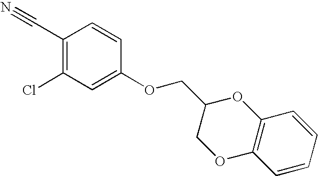 | 108 | (a) |
| 70 | 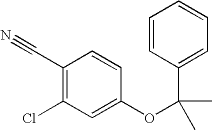 | 53 | (a) |
| 71 | 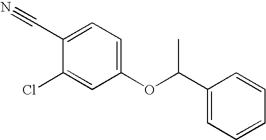 | 13 | (a) |
| 71A | 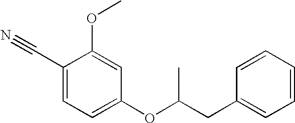 | 306 | (a) |
| 71B | 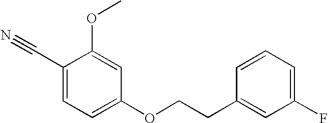 | 155 | (a) |
| 72 | 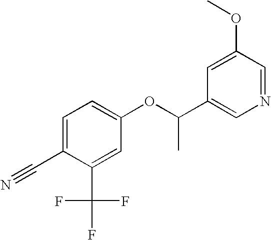 | 203 | (a) |
| 73 | 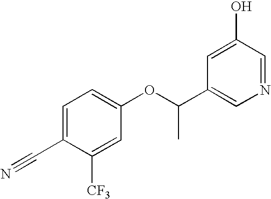 | 110 | (a) |
| 74 | 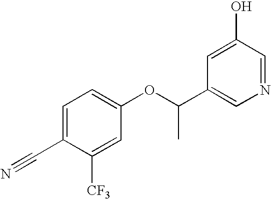 | 35 | (n = 1) |
| 75 | 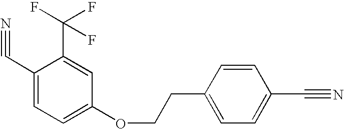 | 343 | (c) |
| 76 | 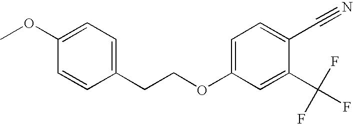 | 281 | (a) |
| 77 | 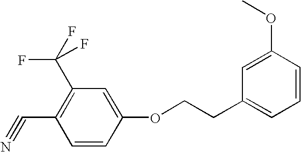 | 109 | (c) |
| 78 | 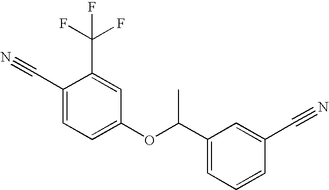 | 37nM | (a) |
| 79 | 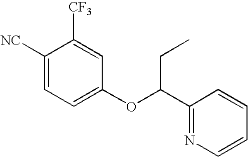 | 148 | |
| 80 | 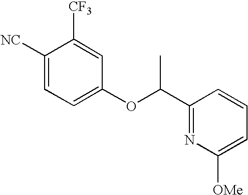 | 120 | (b) |
| 81 | 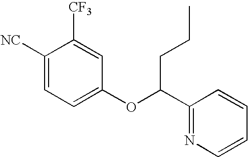 | 54.3 | (a) |
| 82 | 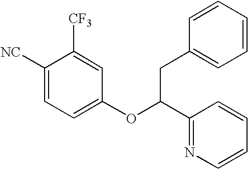 | 238 | (a) |
| 83 | 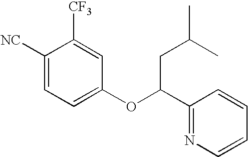 | 119 | (a) |
| 84 | 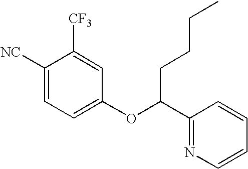 | 161 | (a) |
| 85 | 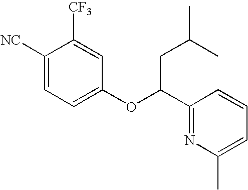 | 167 | (a) |
| 86 | 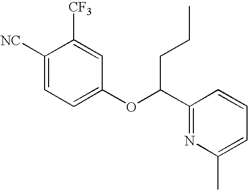 | 330 | (a) |
| 87 | 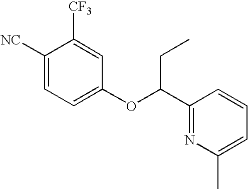 | 483 | (a) |
| 88 | 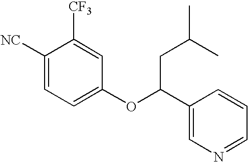 | 218 | |
| 89 | 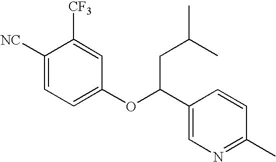 | 137 | |
| 90 | 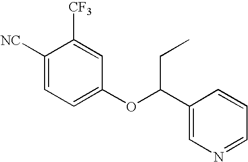 | 167 | |
| 91 | 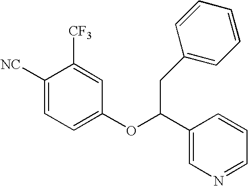 | 116 | |
| 92 | 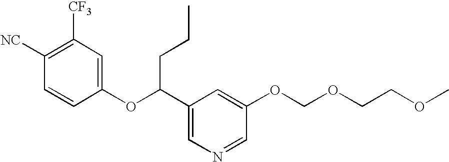 | 74 | |
| 93 | 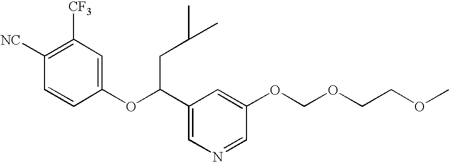 | 98 | |
| 94 | 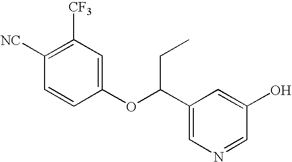 | 75 | |
| 95 | 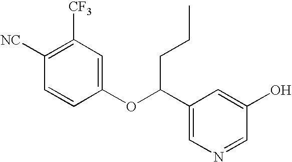 | 68 | (a) |

a—mean of 2 tests
b—mean of 3 tests
c—mean of 4 tests
d—mean of 8 tests
ND—not determined
UA—unavailable

### Example 97

The compounds ability to antagonize the effects of androgen on the androgen receptor were determined in a whole cell assay as described immediately below.

### Experimental Procedure for AR Antagonist Cell Assay

Cell line: MDA-MB453-MMTV clone 54-19. This cell line is a stable transfected cell line with MDA-MB453 cell background (a human breast tumor cell line expressing androgen receptor). A MMTV minimal promoter containing ARE was first cloned in front of a firefly luciferase reporter gene. Then the cascade was cloned into transfection vector pUV120puro. Electroporation method was used for transfecting MDA-MB-453 cell. Puromycin resistant stable cell line was selected.

### Cell Culture Media and Reagents:

Culture medium: DMEM (high glucose, Gibco cat #: 11960-044), 10% FBS, and 1% L-glutamine

Plating medium: DMEM (phenol red free), 10% charcoal treated HyClone serum, 1% L-glutamine

Assay medium: DMEM (phenol red free), 1% charcoal treated HyClone serum, 1% L-glutamine, and 1% penicillin/streptomycin

3× luciferase buffer: 2% beta-mercaptoethanol, 0.6% ATP, 0.0135% luciferine in cell lysis buffer

### Assay Procedure:

Cells are maintained in culture medium, splitting cells when they reach 80-90% confluence.
To test compounds, 10,000 cells/well are plated to opaque 96 cell culture plate in 100 ul/well plating medium, culture for overnight at 37° C. in cell culture incubator. Carefully remove plating medium, then add 80 ul/well of pre-warmed assay medium, add 10 ul/well testing compound (final concentration at) 1000 nM, 200 nM, 40 nM, 8 nM, 1.6 nM, and 0.32 nM), incubate at 37° C. for 30 minutes.
Add 10 ul/well freshly prepared DHT (final concentration at 100 pM) to each well, incubate at 37° C. for 17 hr (overnight).
Add 50 ul/well 3× luciferase buffer, incubate at room temperature for 5 minutes, then count on Luminometer.
The fold induction over background by 100 pM DHT in the absence of testing compounds is standardized as 100% and experimental result is expressed as percentage of inhibition by testing compounds.

The results are described below in Table III. The results are reported as the mean of multiple tests as described below (the numbers of tests are indicated in the footnote). ND denotes that the compound was not tested.

TABLE III

| | | AR Cell |
|---|---|---|
| Example | | Assay |
| # | | IC₅₀ (nM) |
| 1 | 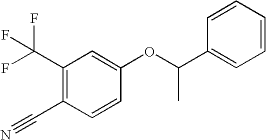 | 90 (c) |
| 2 | 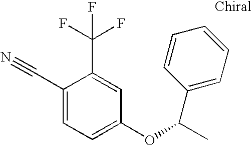 | 381 (a) |
| 3 | 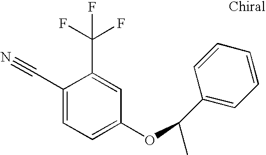 | 61 (a) |
| 4 | 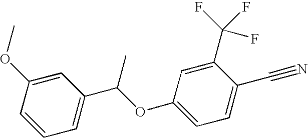 | >1,000 (c) |
| 5 | 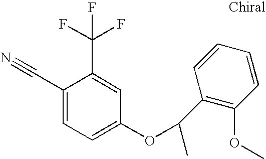 | >1,000 (a) |
| 6 | 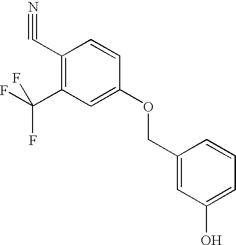 | 6 (a) |
| 7 | 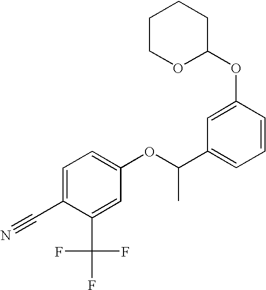 | ND |
| 8 | 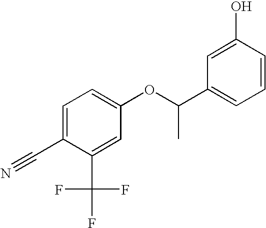 | 529 (a) |
| 9 | 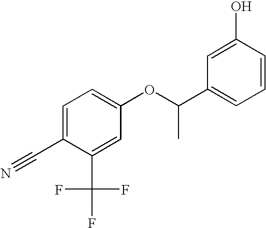 | 13 (c) |
| 10 | 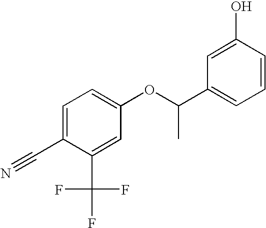 | 1,000 (a) |
| 11 | 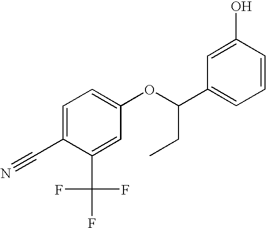 | >1,000 (a) |
| 12 | 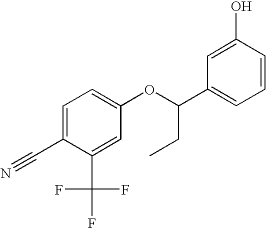 | >1,000 (a) |
| 13 | 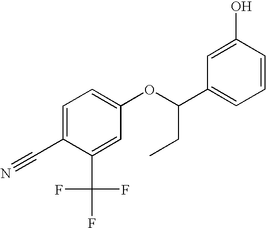 | 31 (a) |
| 14 | 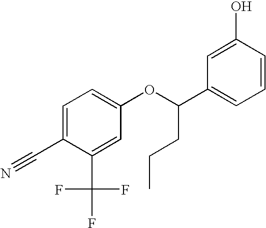 | 36 (c) |
| 15 | 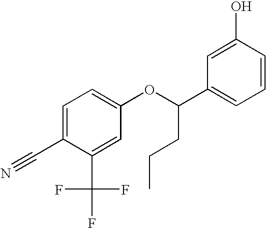 | 102 (b) |
| 16 | 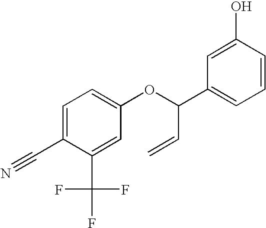 | 416 (a) |
| 17 | 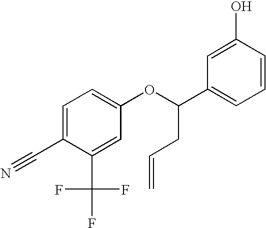 | 105 (a) |
| 18 | 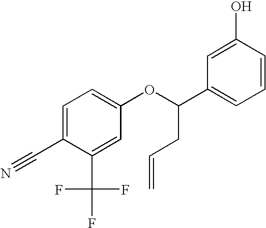 | 86 (a) |
| 19 | 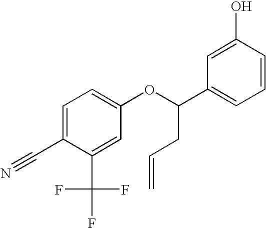 | 34 (c) |
| 20 | 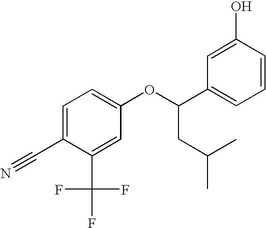 | 40 (c) |
| 21 | 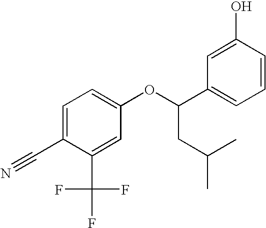 | 79 (a) |
| 22 | 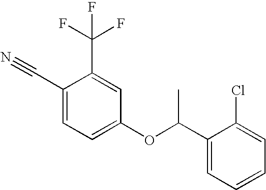 | 102 (a) |
| 23 | 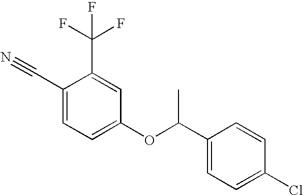 | ND |
| 24 | 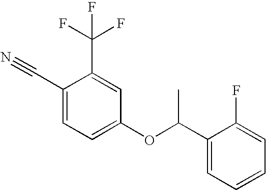 | 826 (a) |
| 25 | 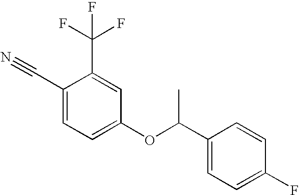 | 92 (a) |
| 26 | 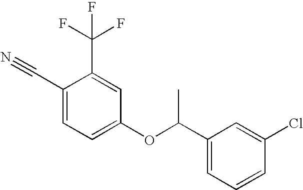 | 56 (a) |
| 27 | 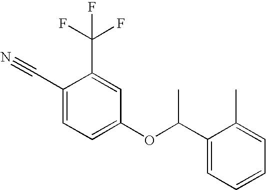 | ND |
| 28 | 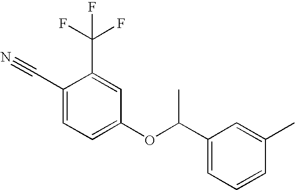 | 573 (a) |
| 29 | 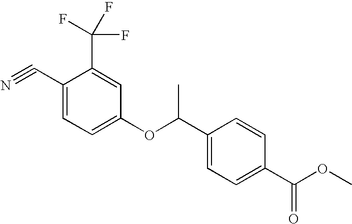 | 132 (a) |
| 30 | 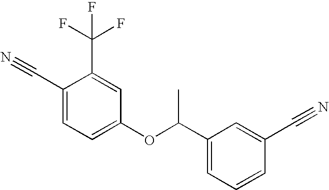 | 7 (a) |
| 31 | 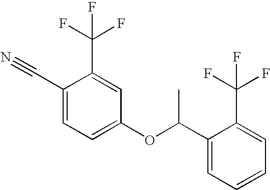 | 398 (a) |
| 32 | 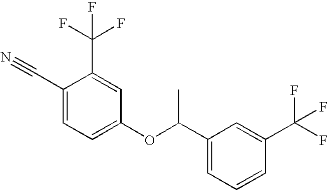 | ND |
| 33 | 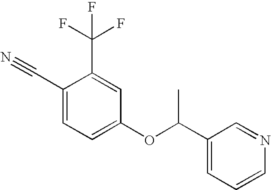 | 63 (c) |
| 34 | 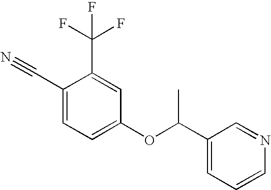 | 94 (c) |
| 35 | 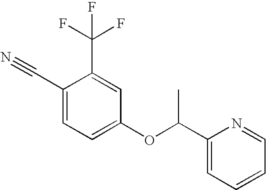 | 254 (a) |
| 36 | 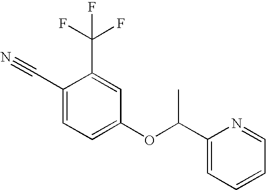 | 28 (a) |
| 37 | 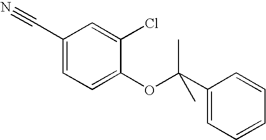 | >1,000 (a) |
| 38 | 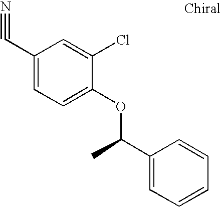 | ND |
| 39 | 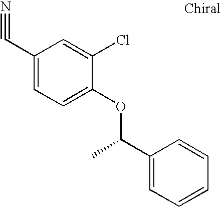 | ND |
| 40 | 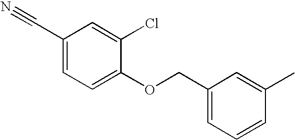 | ND |
| 41 | 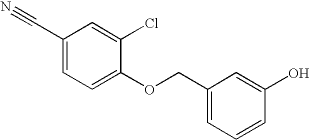 | 286 (a) |
| 42 | 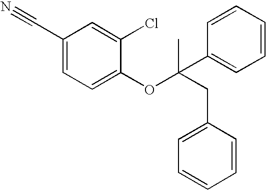 | ND |
| 43 | 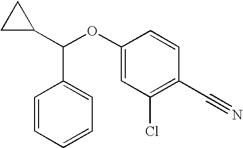 | ND |
| 44 | 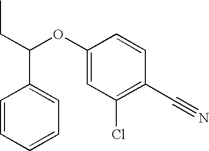 | ND |
| 45 | 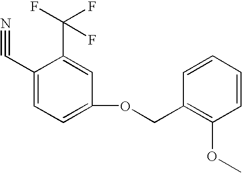 | >1,000 (a) |
| 46 | 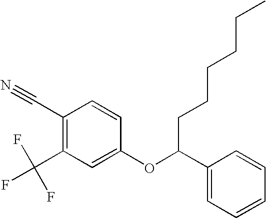 | >1,000 (a) |
| 47 | 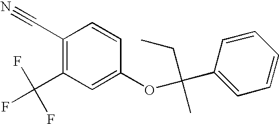 | ND |
| 48 | 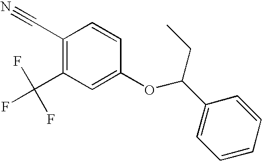 | 345 (a) |
| 50 | 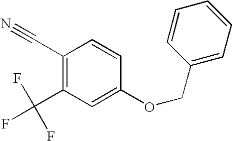 | 508 (a) |
| 51 | 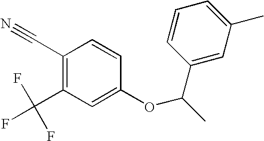 | >1,000 (a) |
| 52 | 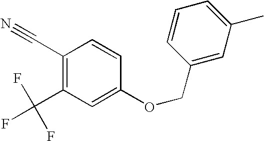 | ND |
| 53 | 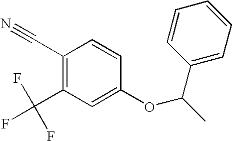 | 55 (a) |
| 54 | 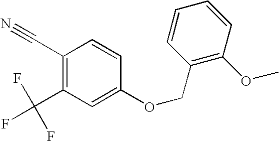 | ND |
| 55 | 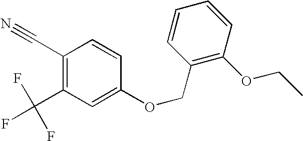 | >1,000 (a) |
| 56 | 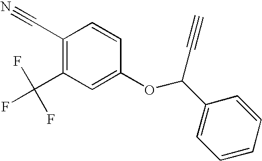 | 20 (a) |
| 57 | 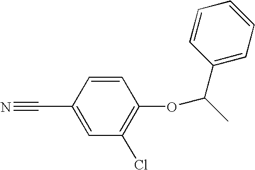 | 86 (a) |
| 58 | 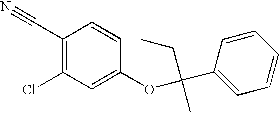 | >1,000 (a) |
| 59 | 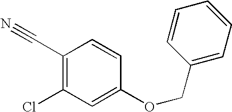 | 108 (a) |
| 60 | 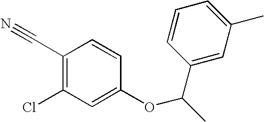 | >1,000 (a) |
| 61 | 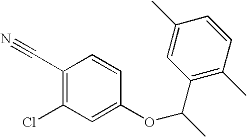 | >1,000 (a) |
| 62 | 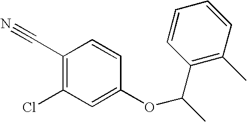 | 464 (a) |
| 63 | 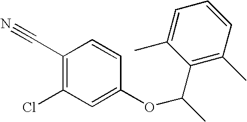 | ND |
| 64 | 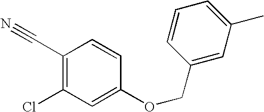 | >1,000 (a) |
| 65 | 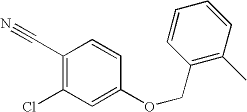 | >1,000 (c) |
| 66 | 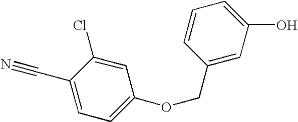 | 4 (a) |
| 67 | 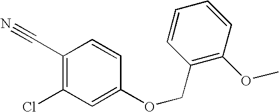 | 166 (a) |
| 68 | 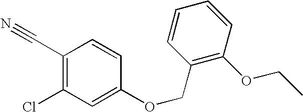 | >1,000 (a) |
| 69 | 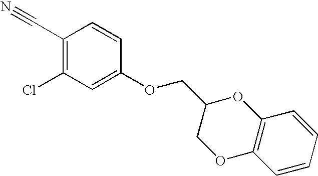 | >1,000 (a) |
| 70 | 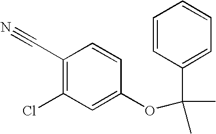 | >1,000 (a) |
| 71 | 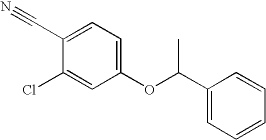 | 8 (a) |
| 71A | 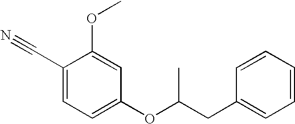 | ND |
| 71B | 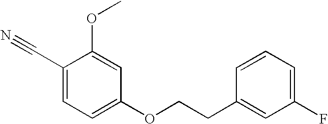 | 41.4 (a) |
| 72 | 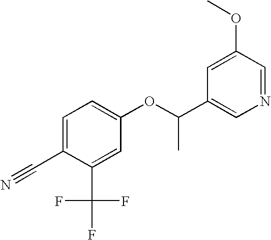 | 441 (a) |
| 73 | 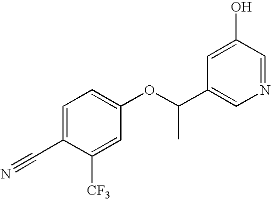 | 70 (c) |
| 74 | 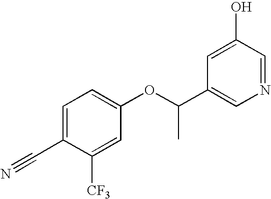 | 111 (a) |
| 75 | 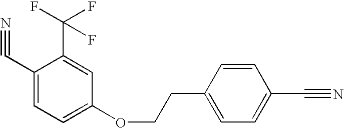 | 55 (a) |
| 76 | 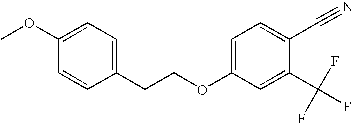 | ND |
| 77 | 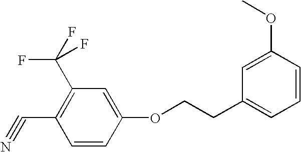 | 11 (c) |
| 78 | 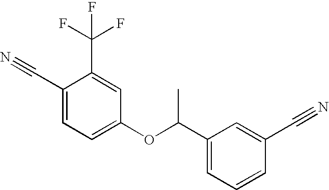 | 47 nM (a) |
| 79 | 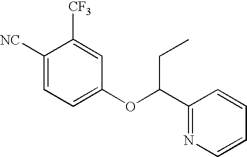 | 27.1 |
| 80 | 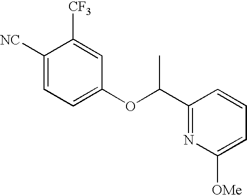 | 152 (a) |
| 81 | 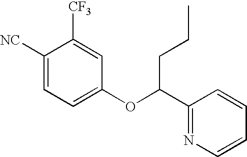 | 280 (a) |
| 82 | 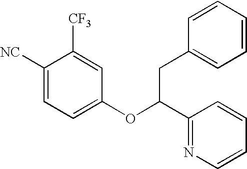 | >1000 (a) |
| 83 | 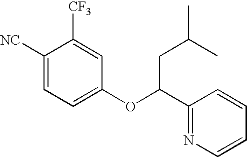 | >1000 (a) |
| 84 | 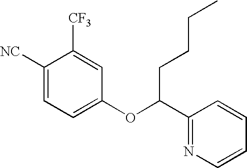 | >667 (a) |
| 85 | 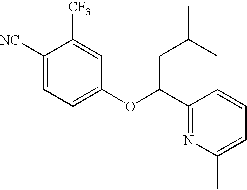 | >1000 (a) |
| 86 | 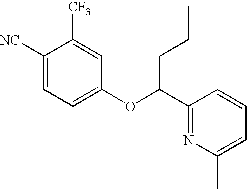 | ND |
| 87 | 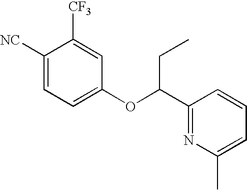 | ND |
| 88 | 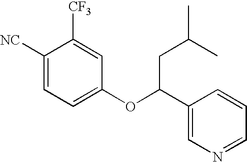 | 50 (d) |
| 89 | 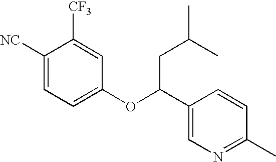 | 56 (a) |
| 90 | 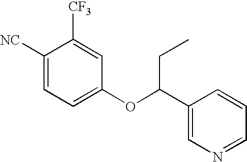 | 142 (a) |
| 91 | 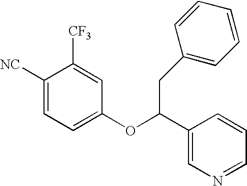 | 342 (a) |
| 92 | 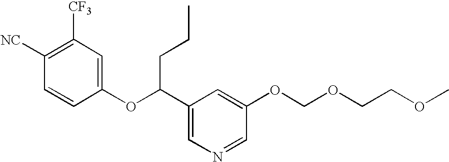 | >934 (a) |
| 93 | 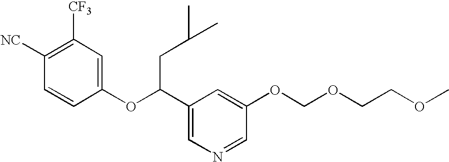 | 202 (a) |
| 94 | 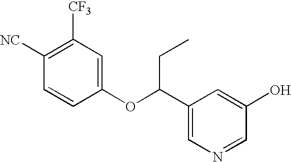 | 202 (a) |
| 95 | 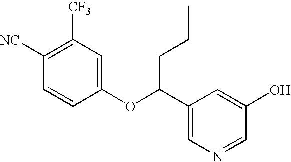 | 37 (c) |

a—mean of 2 tests
b—mean of 3 tests
c—mean of 4 tests
d—mean of 8 tests
ND—not determined
UA—unavailable

### Example 98

### Animal Model for Inhibition of Sebum Production

Luderschmidt et al. describes an animal model for testing whether compounds are capable of modulating sebum secretion, Arch. Derm. Res., 258, 185-191 (1977). This model uses male Syrian hamsters, whose ears contain sebaceous glands. Based on binding data and cellular assay data, selected compounds were chosen for screening in this model. Those compounds included the products of Examples 1, 20, 81, 82, and 109.

Testing for sebum inhibition was carried out in the following manner. Male Syrian hamsters aged 9 to 10 weeks were introduced into the laboratory environment and acclimated for 2 weeks prior to use in the study. Each group consisted of 5 animals and run in parallel with vehicle and positive controls. Prior to administration, a sufficient quantity of each compound was dissolved in 1 mL of a solvent consisting of ethanol, transcutol, and propylene glycol (60120120% v/v/v) to achieve the final concentration specified in Table IV below.

Animals were dosed topically twice daily, five days a week, for 4 weeks. Each dose consisted of 25 micro liters of vehicle control or drug. The dose was applied to the ventral surfaces of both the right and left ears. All animals were sacrificed approximately 18-24 hours after the final dose. The right ears were collected from each animal and used for sebum analysis.

The ears were prepped for HPLC analysis in the following manner. One 8 mm distal biopsy punch was taken, just above the anatomical “V” mark in the ear to normalize the sample area. The punch was pulled apart. The ventral biopsy surface (the area where the topical dose was directly applied to the sebaceous glands) was retained for testing and the dorsal surface of the biopsy punch was discarded.

Tissue samples were blown with N₂ gas and stored at −80° C. under nitrogen until HPLC analysis. In addition to ear samples, an aliquot of each drug and vehicle (at least 250 ul) was also stored at −80° C. for inclusion in the HPLC analysis.

HPLC analysis was carried out on an extract of the tissue sample. Tissue samples were contacted with 3 ml of solvent (a 4:1 admixture of 2,2,4-trimethylpentane and isopropyl alcohol). The mixture was shaken for 15 minutes and stored overnight at room temperature, protected from light. The next morning 1 milliliter of water was added to the sample and shaken for 15 minutes. The sample was then centrifuged at approximately 1500 rpm for 15 minutes. Two ml of the organic phase (top layer) was transferred to a glass vial, dried at 37° C., under nitrogen, for approximately 1 hour, and then lyophilized for approximately 48 hours. The samples were then removed from the lyophilizer and each vial was reconstituted with 600 μl of solvent A (trimethylpentane/tetrahydrofuran (99:1)). The samples were then recapped and vortexed for 5 minutes.

Two hundred (200) μl of each sample was then transferred to a pre-labeled 200 μl HPLC vial with 200 μL glass inserts. The HPLC vials were placed in the autosampler tray for the Agilent 1100 series HPLC unit. The Agilent 1100 HPLC system consisted of a thermostated autosampler, a quarternary pump, a column heater, and an A/D interface module. All components were controlled by Agilent ChemStation software. A Waters Spherisorb S3W 4.6×100 mm analytical column was maintained at 30° C. by the Agilent column heater unit.

The HPLC autosampler was programmed to maintain the sample temperature at 20° C. throughout the run.

Ten (10) uL of each sample was injected in triplicate into the column. Two solvents were used for the solvent gradient. Solvent A was an admixture of trimethylpentane and tetrahydrofuran (99:1). Solvent B was ethylacetate. The gradient utilized is described in the table below:

TABLE IV

| | | | Flow |
|---|---|---|---|
| Time (min) | Solv A (%) | Solv B (%) | (mL/min) |
| 0 | 99 | 1 | 2 |
| 2 | 96 | 4 | 2 |
| 6 | 60 | 40 | 2 |
| 7 | 5 | 95 | 2 |
| 10 | 5 | 95 | 2 |
| 10.1 | 99 | 1 | 2 |

The Sedex 75 Evaporative Light Scattering Detector (ELSD) was operated at 45° C. with a gain of 5, and N₂ pressure maintained at 3.1 bar. Analog signal obtained by the instrument was sent to the Agilent A/D interface module where it was converted to a digital output. The conversion was based on a 10000 mAU/volt set point and the data rate was set at 10 Hz (0.03 min). The resulting digital output was then feed into the Agilent ChemStation software for integration of the peak area.

The results of the HPLC analysis are reported below in Table V. The results are reported as the reduction in cholesterol ester (CE) and wax ester (WE) production, when compared to the vehicle control. A negative value reflects an increase in sebum, whereas a positive reflects a decrease.

TABLE V

| | % CE | % WE | Sum of | Concen. |
|---|---|---|---|---|
| Example # | reduction | reduction | WE & CE | Tested |
| 1 | 52 | 71 | 123 | 3% |
| 6 | 25 | 40 | 65 | 1.5% |
| 9 | 38 | 52 | 90 | 3% |
| 14 | 18 | 38 | 56 | 3% |
| 15 | −10 | −13 | −23 | 1% |
| 19 | 6 | 13 | 19 | 1% |
| 21 | 7 | 19 | 26 | 1% |
| 74 | 21 | 24 | 45 | 1% |
| 79 | 20 | 27 | 47 | 1% |
| 80 | 39 | 50 | 89 | 1% |
| 88 | 17 | 25 | 42 | 1% |
| 89 | 21 | 35 | 56 | 1% |

What is claimed is:

1. A compound of the formula: 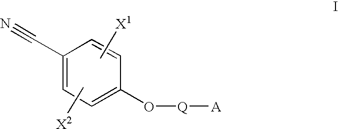
or a salt or solvate thereof,
in which;
a) X¹ is represented by halogen, cyano, NO₂, C₁-C₆ alkyl, C₁-C₆ alkoxy or haloalkyl;
b) X² is represented by hydrogen, halogen, cyano, NO₂, C₁-C₆ alkyl, C₁-C₆ alkoxy or haloalkyl;
c) A is represented by: 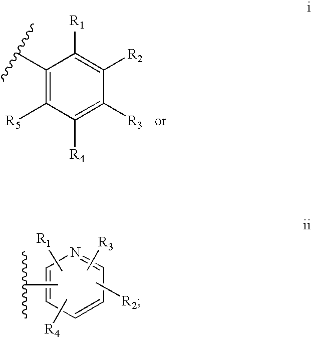
d) Q is represented by C₁-C₆ alkylene which is unsubstituted or optionally substituted with one or more groups each independently selected from:
i) C₁-C₆ alkyl, optionally substituted;
ii) C₂-C₆ alkenyl, optionally substituted;
iii) C₂-C₆ alkynyl, optionally substituted;
iv) C₃-C₆ cycloalkyl, optionally substituted;
v) —(C₁-C₆) alkyl(C₆-C₁₀) aryl, in which the alkyl and aryl moieties may each be optionally substituted;
vi) —(C₆-C₁₀) aryl(C₁-C₆) alkyl, in which the alkyl and aryl moieties may each be optionally substituted; and
vii) C₁-C₆ alkoxy, optionally substituted;
e) R₁, R₂, R₃, R₄ and R₅ are each independently represented by a substituent selected from the group consisting of:
i) hydrogen;
ii) halogen;
iii) hydroxyl;
iv) amino;
v) nitro;
vi) cyano;
vii) (C₁-C₁₂)alkyl, optionally substituted;
viii) (C₁-C₆)alkoxy, optionally substituted;
ix) (C₃-C₆)cycloalkoxy, optionally substituted;
x) (C₁-C₃)haloalkyl, optionally substituted;
xi) (C₂-C₁₂)alkenyl, optionally substituted;
xii) (C₂-C₁₂)alkynyl, optionally substituted;
xiii) (C₃-C₁₀)cycloalkyl, optionally substituted;
xiv) (C₆-C₁₀)aryl, optionally substituted,
xv) (C₆-C₁₀)aryl(C₁-C₆)alkyl, in which the alkyl and aryl moieties may each be optionally substituted,
xvi) heteroaryl, optionally substituted;
xvii) heteroaryl(C₁-C₁₂)alkyl, in which the heteroaryl and alkyl moieties may each be optionally substituted;
xviii) —O-heterocyclic, optionally substituted;
xix) heterocyclic(C₁-C₁₂)alkyl-O—, in which the alkyl and heterocyclic moieties may each be optionally substituted;
xx) —CO₂R₆;
xxi) —O—COR₆;
xxii) —CONHR₆;
xxiii) —NCOP6; and
xxiv) —O—(C₁-C₆)alkyl-O—(C₁-C₆)alkyl-O—(C₁-C₆)alkyl; and
f) R₆ is independently hydrogen or C₁-C₆ alkyl;
however, when A is represented by formula i, X¹ or X² is halogen, and Q is methylene, ethylene or n-propylene, A is not 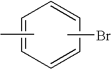
and R₃ is not cyano, bromine, alkynyl, or halogen.

2. A compound according to claim 1 in which X² is hydrogen.

3. A compound according to claim 1 in which X² is hydrogen and X¹ is selected from trifluoromethyl, halogen and C₁-C₆ alkoxy.

4. A compound according to claim 1 in which A is

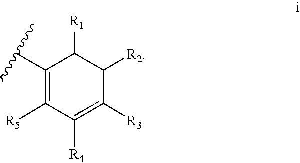

5. A compound according to claim 1 in which A is

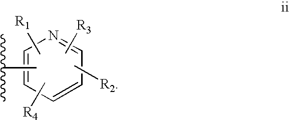

6. A compound according to claim 1 in which Q is C₁-C₆ alkylene, optionally substituted.

7. A compound according to claim 6 in which Q is selected from methylene, ethylene and propylene.

8. A compound according to claim 4 in which X¹ is represented by halogen, C₁-C₆ alkoxy or halo alkyl, X² is hydrogen, Q is methylene and R₁, R₂, R₃, R₄ and R₅ are independently selected from hydrogen and hydroxy.

9. A compound selected from the group comprising
4-(1-phenyl-ethoxy)-2-trifluoromethyl-benzonitrile;
(S)-4-(1-phenyl-ethoxy)-2-trifluoromethyl-benzonitrile;
(R)-4-(1-phenyl-ethoxy)-2-trifluoromethyl-benzonitrile;
4-[1-(2-methoxy-phenyl)-ethoxy]-2-t-trifluoromethyl-benzonitrile;
4-[(3-hydroxybenzyl)oxy]-2-(trifluoromethyl)benzonitrile;
4-[1-(3-hydroxyphenyl)ethoxy]-2-(trifluoromethyl)benzonitrile;
(−)-4-[1-(3-hydroxyphenyl)ethoxy]-2-(trifluoromethyl)benzonitrile;
(+)-4-[1-(3-hydroxyphenyl)ethoxy]-2-(trifluoromethyl)benzonitrile;
4-(1-Pyridin-3-yl-ethoxy)-2-trifluoromethyl-benzonitrile;
4-(1-Pyridin-2-yl-ethoxy)-2-trifluoromethyl-benzonitrile;
4-(1-Pyridin-3-yl-ethoxy)-2-trifluoromethyl-benzonitrile;
4-[1-(5-hydroxypyridin-3-yl)ethoxy]-2-(trifluoromethyl)benzonitrile;
(+)4-[1-(5-hydroxypyridin-3-yl)ethoxy]-2-(trifluoromethyl)benzonitrile, or a pharmaceutically acceptable salt thereof.

10. (canceled)

11. A method for inhibiting activation of the androgen receptor comprising the administration of a compound according to claim 1 to a patient in need thereof.

12. A method for alleviating a condition selected from the group consisting of hormone dependent cancers, benign hyperplasia of the prostate, acne, hirsutism, excess sebum, alopecia, premenstrual syndrome, lung cancer, precocious puberty, osteoporosis, hypogonadism, age-related decrease in muscle mass, and anemia comprising the administration of a compound according to claim 1 to a patient in need thereof.

13. A pharmaceutical composition comprising a compound according to claim 1 in admixture with one or more pharmaceutically acceptable excipients.

14. A topical pharmaceutical formulation comprising a compound according to claim 1 in admixture with or more pharmaceutically acceptable excipients suitable for dermal application.

15. (canceled)
